(19) United States
(12) Patent Application Publication
(10) Pub. No.: US 2025/0017530 A1
(43) Pub. Date: Jan. 16, 2025

(54) Motion Recognition Clothing (Wearable Device for Measuring Full-Body Configuration and Motion)

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 18/900,784

(22) Filed: Sep. 29, 2024

Related U.S. Application Data
(63) Continuation-in-part of application No. 18/369,129, filed on Sep. 15, 2023.
Application No. 18/369,129 is a continuation-in-part of application No. 17/721,866, filed on Apr. 15, 2022, now Pat. No. 11,892,286.
Application No. 17/721,866 is a continuation-in-part of application No. 17/356,377, filed on Jun. 23, 2021, now Pat. No. 11,304,628.
Application No. 17/356,377 is a continuation-in-part of application No. 16/751,245, filed on Jan. 24, 2020, now Pat. No. 11,071,498.
Application No. 16/751,245 is a continuation-in-part of application No. 16/543,056, filed on Aug. 16, 2019, now Pat. No. 10,839,202.
Application No. 16/543,056 is a continuation-in-part of application No. 16/017,439, filed on Jun. 25, 2018, now Pat. No. 10,921,886.
Application No. 16/017,439 is a continuation-in-part of application No. 16/010,448, filed on Jun. 16, 2018, now Pat. No. 10,602,965.
Application No. 16/010,448 is a continuation-in-part of application No. 15/702,081, filed on Sep. 12, 2017, now Pat. No. 10,716,510.
Application No. 15/702,081 is a continuation-in-part of application No. 16/010,448, filed on Jun. 16, 2018, now Pat. No. 10,602,965.
Application No. 16/017,439 is a continuation-in-part of application No. 16/010,448, filed on Jun. 16, 2018, now Pat. No. 10,602,965.
Application No. 16/010,448 is a continuation-in-part of application No. 15/702,081, filed on Sep. 12, 2017, now Pat. No. 10,716,510.
Application No. 15/702,081 is a continuation-in-part of application No. 15/227,254, filed on Aug. 3, 2016, now Pat. No. 10,321,873.
Application No. 15/227,254 is a continuation-in-part of application No. 15/227,254, filed on Aug. 3, 2016, now Pat. No. 10,321,873.
Application No. 15/227,254 is a continuation-in-part of application No. 14/795,373, filed on Jul. 9, 2015, now abandoned.
Application No. 14/795,373 is a continuation-in-part of application No. 15/130,995, filed on Apr. 17, 2016, now Pat. No. 9,891,718.
Application No. 15/130,995 is a continuation-in-part of application No. 15/079,447, filed on Mar. 24, 2016, now Pat. No. 10,234,934.
Application No. 15/079,447 is a continuation-in-part of application No. 14/736,652, filed on Jun. 11, 2015, now abandoned.
Application No. 14/736,652 is a continuation-in-part of application No. 14/664,832, filed on Mar. 21, 2015, now Pat. No. 9,582,072.
Application No. 14/664,832 is a continuation-in-part of application No. 14/664,832, filed on Mar. 21, 2015, now Pat. No. 9,582,072.
Application No. 14/664,832 is a continuation-in-part of application No. 14/463,741, filed on Aug. 20, 2014, now Pat. No. 9,588,582.
Application No. 14/463,741 is a continuation-in-part of application No. 14/736,652, filed on Jun. 11, 2015, now abandoned.
Application No. 14/736,652 is a continuation-in-part of application No. 14/664,832, filed on Mar. 21, 2015, now Pat. No. 9,582,072.
Application No. 14/664,832 is a continuation-in-part of application No. 14/463,741, filed on Aug. 20, 2014, now Pat. No. 9,588,582.
(60) Provisional application No. 62/797,266, filed on Jan. 26, 2019.
(60) Provisional application No. 62/797,266, filed on Jan. 26, 2019.
(60) Provisional application No. 62/727,798, filed on Sep. 6, 2018.
(60) Provisional application No. 62/683,237, filed on Jun. 11, 2018.
(60) Provisional application No. 62/683,237, filed on Jun. 11, 2018.
(60) Provisional application No. 62/538,793, filed on Jul. 30, 2017.
(60) Provisional application No. 62/538,793, filed on Jul. 30, 2017.
(60) Provisional application No. 62/449,735, filed on Jan. 24, 2017.
(60) Provisional application No. 62/357,957, filed on Jul. 2, 2016.
(60) Provisional application No. 62/150,886, filed on Apr. 22, 2015.
(60) Provisional application No. 62/150,886, filed on Apr. 22, 2015.
(60) Provisional application No. 62/187,906, filed on Jul. 2, 2015.
(60) Provisional application No. 62/182,473, filed on Jun. 20, 2015.
(60) Provisional application No. 62/086,053, filed on Dec. 1, 2014.
(60) Provisional application No. 62/065,032, filed on Oct. 17, 2014.
(60) Provisional application No. 62/014,747, filed on Jun. 20, 2014.
(60) Provisional application No. 61/976,650, filed on Apr. 8, 2014.
(60) Provisional application No. 61/878,893, filed on Sep. 17, 2013.

(51) Int. Cl. A61B 5/00

(52) U.S. Cl. CPC A61B 5/6804

(57) ABSTRACT

Motion recognition clothing is smart clothing which measures changes in a person's body configuration and motion. In an example, a combination of upper-body and lower-body motion recognition clothing can measure changes in full-body configuration and motion. Selected body joints are each spanned by a plurality of flexible energy pathways. Each flexible energy pathway is in communication with an energy emitter and an energy receiver. Body joint movements deform the energy pathways, this deformation changes energy transmission through the energy pathways, and changes in energy transmission are then analyzed to measure body configuration and motion.

### CROSS-REFERENCE TO RELATED APPLICATIONS

This application is a continuation in part of patent application Ser. No. 18/369,129 filed on 2023 Sep. 15.

Patent application Ser. No. 18/369,129 was a continuation in part of patent application Ser. No. 17/721,866 filed on 2022 Apr. 15 which issued as U.S. Pat. No. 11,892,286 on 2024 Feb. 6. Patent application Ser. No. 17/721,866 was a continuation in part of patent application Ser. No. 17/356,377 filed on 2021 Jun. 23 which issued as U.S. Pat. No. 11,304,628 on 2022 Apr. 19. Patent application Ser. No. 17/721,866 was a continuation in part of patent application Ser. No. 17/356,377 filed on 2021 Jun. 23 which issued as U.S. Pat. No. 11,304,628 on 2022 Apr. 19. Patent application Ser. No. 17/356,377 was a continuation in part of patent application Ser. No. 16/751,245 filed on 2020 Jan. 24 which issued as U.S. Pat. No. 11,071,498 on 2021 Jul. 27.

Patent application Ser. No. 16/751,245 was a continuation in part of patent application Ser. No. 16/543,056 filed on 2019 Aug. 16 which issued as U.S. Pat. No. 10,839,202 on 2020 Nov. 17. Patent application Ser. No. 16/751,245 claimed the priority benefit of patent provisional application 62/797,266 filed on 2019 Jan. 26. Patent application Ser. No. 16/751,245 was a continuation in part of patent application Ser. No. 16/017,439 filed on 2018 Jun. 25 which issued as U.S. Pat. No. 10,921,886 on 2020 Feb. 16. Patent application Ser. No. 16/751,245 was a continuation in part of patent application Ser. No. 16/010,448 filed on 2018 Jun. 16 which issued as U.S. Pat. No. 10,602,965 on 2020 Mar. 31. Patent application Ser. No. 16/751,245 was a continuation in part of patent application Ser. No. 15/702,081 filed on 2017 Sep. 12 which issued as U.S. Pat. No. 10,716,510 on 2020 Jul. 21.

Patent application Ser. No. 16/543,056 claimed the priority benefit of patent provisional application 62/797,266 filed on 2019 Jan. 26. Patent application Ser. No. 16/543,056 claimed the priority benefit of patent provisional application 62/727,798 filed on 2018 Sep. 6. Patent application Ser. No. 16/543,056 was a continuation in part of patent application Ser. No. 16/010,448 filed on 2018 Jun. 16 which issued as U.S. Pat. No. 10,602,965 on 2020 Mar. 31. Patent application Ser. No. 16/017,439 was a continuation in part of patent application Ser. No. 16/010,448 filed on 2018 Jun. 16 which issued as patent Ser. No. 10/602,965 on 2020 Mar. 31. Patent application Ser. No. 16/017,439 claimed the priority benefit of patent provisional application 62/683,237 filed on 2018 Jun. 11. Patent application Ser. No. 16/017,439 was a continuation in part of patent application Ser. No. 14/795,373 filed on 2015 Jul. 9.

Patent application Ser. No. 16/010,448 claimed the priority benefit of patent provisional application 62/683,237 filed on 2018 Jun. 11. Patent application Ser. No. 16/010,448 was a continuation in part of patent application Ser. No. 15/702,081 filed on 2017 Sep. 12 which issued as U.S. Pat. No. 10,716,510 on 2020 Jul. 21. Patent application Ser. No. 16/010,448 claimed the priority benefit of patent provisional application 62/538,793 filed on 2017 Jul. 30. Patent application Ser. No. 16/010,448 was a continuation in part of patent application Ser. No. 15/227,254 filed on 2016 Aug. 3 which issued as U.S. Pat. No. 10,321,873 on 2019 Jun. 18.

Patent application Ser. No. 15/702,081 claimed the priority benefit of patent provisional application 62/538,793 filed on 2017 Jul. 30. Patent application Ser. No. 15/702,081 claimed the priority benefit of patent provisional application 62/449,735 filed on 2017 Jan. 24. Patent application Ser. No. 15/702,081 was a continuation in part of patent application Ser. No. 15/227,254 filed on 2016 Aug. 3 which issued as U.S. Pat. No. 10,321,873 on 2019 Jun. 18. Patent application Ser. No. 15/702,081 was a continuation in part of patent application Ser. No. 14/795,373 filed on 2015 Jul. 9.

Patent application Ser. No. 15/227,254 claimed the priority benefit of patent provisional application 62/357,957 filed on 2016 Jul. 2. Patent application Ser. No. 15/227,254 was a continuation in part of patent application Ser. No. 15/130,995 filed on 2016 Apr. 17 which issued as U.S. Pat. No. 9,891,718 on 2018 Feb. 13. Patent application Ser. No. 15/227,254 was a continuation in part of patent application Ser. No. 15/079,447 filed on 2016 Mar. 24 which issued as U.S. Pat. No. 10,234,934 on 2019 Mar. 19. Patent application Ser. No. 15/227,254 was a continuation in part of patent application Ser. No. 14/736,652 filed on 2015 Jun. 11. Patent application Ser. No. 15/227,254 was a continuation in part of patent application Ser. No. 14/664,832 filed on 2015 Mar. 21 which issued as U.S. Pat. No. 9,582,072 on 2017 Feb. 28.

Patent application Ser. No. 15/130,995 claimed the priority benefit of patent provisional application 62/150,886 filed on 2015 Apr. 22. Patent application Ser. No. 15/079,447 claimed the priority benefit of patent provisional application 62/150,886 filed on 2015 Apr. 22. Patent application Ser. No. 15/079,447 was a continuation in part of patent application Ser. No. 14/664,832 filed on 2015 Mar. 21 which issued as U.S. Pat. No. 9,582,072 on 2017 Feb. 28. Patent application Ser. No. 15/079,447 was a continuation in part of patent application Ser. No. 14/463,741 filed on 2014 Aug. 20 which issued as U.S. Pat. No. 9,588,582 on 2017 Mar. 7.

Patent application Ser. No. 14/795,373 claimed the priority benefit of patent provisional application 62/187,906 filed on 2015 Jul. 2. Patent application Ser. No. 14/795,373 claimed the priority benefit of patent provisional application 62/182,473 filed on 2015 Jun. 20. Patent application Ser. No. 14/795,373 was a continuation in part of patent application Ser. No. 14/736,652 filed on 2015 Jun. 11. Patent application Ser. No. 14/795,373 claimed the priority benefit of patent provisional application 62/086,053 filed on 2014 Dec. 1. Patent application Ser. No. 14/795,373 claimed the priority benefit of patent provisional application 62/065,032 filed on 2014 Oct. 17.

Patent application Ser. No. 14/736,652 was a continuation in part of patent application Ser. No. 14/664,832 filed on 2015 Mar. 21 which issued as U.S. Pat. No. 9,582,072 on 2017 Feb. 28. Patent application Ser. No. 14/736,652 claimed the priority benefit of patent provisional application 62/014,747 filed on 2014 Jun. 20. Patent application Ser. No. 14/664,832 was a continuation in part of patent application Ser. No. 14/463,741 filed on 2014 Aug. 20 which issued as U.S. Pat. No. 9,588,582 on 2017 Mar. 7. Patent application Ser. No. 14/664,832 claimed the priority benefit of patent provisional application 61/976,650 filed on 2014 Apr. 8. Patent application Ser. No. 14/463,741 claimed the priority benefit of patent provisional application 61/878,893 filed on 2013 Sep. 17.

The entire contents of these applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to devices for measurement of body configuration and motion.

### Introduction

Wearable devices (e.g. smart watches, fitness bands, and smart glasses) which measure body motion from a single location on a person's body are useful for many applications. They are especially useful for applications which involve relatively-uniform motion of a person's entire body. For example, overall upward and downward motion of a person's wrist, arm, torso, or head can be used as a proxy for steps in walking or running. However, there are many applications and activities in which body motion is complex and non-uniform. These require measurement of full-body configuration and motion.

There are many potential human-to-computer interface applications for measurement of full-body configuration and motion, including: directing character actions or entering commands in virtual reality, computer gaming, or telecommunication; gesture recognition as part of a human-computer interface; telerobotics (e.g. remote surgery) or telepresence; animating a virtual character in a movie; guiding dance instruction or recording dance performance motions; and guiding musical instrument instruction or recording instrument playing motions.

There are many potential sports applications for measurement of full-body configuration and motion, including athletic training, motion capture, sports injury prevention, and performance analysis for sports which involve extensive lower-body motion (such as bicycling and soccer) and complex arm and torso motion (such as golf, basketball, baseball, tennis, and Frisbee). There are many potential health, fitness, and medical applications for measurement of full-body configuration and motion, including: medical condition diagnosis (e.g. gait analysis); range of motion assessment; physical therapy; medical therapy (e.g. rehabilitation) evaluation; posture evaluation and correction; posture-related injury prevention; respiratory function assessment; virtual reality exercise; weight management; ambulatory telerobotics; avoidance of repeated motion injuries; and fall prevention and detection.

For these applications, a method of measuring full-body configuration and motion is needed, including measuring the motions of different body joints and members relative to each other. One method for measuring full-body configuration and motion is camera-based motion capture. However, limitations of camera-based motion capture include: loss of tracking body portions which are visually obscured; and limited mobility to track a person in a distance-traveling activity.

Another method for measuring full-body configuration and motion is attaching multiple motion sensors (e.g. inertial motion units or IMUs) to different parts of a person's body and then computing the relative locations of these sensors in 3D space. However, limitations of multiple motion sensors include: drift between measured and actual sensor location due to compounding spatial-estimation errors; time lag due to spatial computing complexity; and how to attach multiple (current generation) IMUs to clothing in a non-obtrusive manner. Better methods for measurement and modeling of full-body configuration and motion are needed.

### REVIEW OF THE RELEVANT ART

U.S. Pat. No. 6,145,551 (Jayaraman et al., Nov. 14, 2000, “Full-Fashioned Weaving Process for Production of a Woven Garment with Intelligence Capability”) discloses a full-fashioned weaving process for producing a woven garment which can accommodate and include holes. U.S. Pat. No. 6,315,009 (Jayaraman et al., Nov. 13, 2001, “Full-Fashioned Garment with Sleeves Having Intelligence Capability”) discloses a full-fashioned weaving process for the production of a woven garment which can accommodate and include sleeves. U.S. Pat. No. 6,381,482 (Jayaraman et al., Apr. 30, 2002, “Fabric or Garment with Integrated Flexible Information Infrastructure”) discloses a modular electronic garment.

U.S. Pat. No. 6,487,906 (Hock, Dec. 3, 2002, “Flexible Film Sensor System for Monitoring Body Motion”) discloses a sequence of low force, high compliance, long extension, piezofilm-based sensors for a biofeedback system for self-monitoring of selected body motions. U.S. Pat. No. 6,687,523 (Jayaramen et al., Feb. 3, 2004, “Fabric or Garment with Integrated Flexible Information Infrastructure for Monitoring Vital Signs Of Infants”) discloses an infant garment which ensures a snug fit for the baby so that the sensors stay in place to minimize the risk of false alarms.

U.S. Pat. No. 6,970,731 (Jayaraman et al., Nov. 29, 2005, “Fabric-Based Sensor for Monitoring Vital Signs”) discloses a woven or knitted fabric-based sensor for monitoring vital signs or other electrical impulses. U.S. Pat. No. 7,771,318 (Narayanaswami, Aug. 10, 2010, “Device for Monitoring a User's Posture”) and U.S. Pat. No. 7,850,574 (Narayanaswami, Dec. 14, 2010, “Device for Monitoring a User's Posture”) disclose a wearable device which monitors a person's posture. U.S. Pat. No. 7,981,057 (Stewart, Jul. 19, 2011, “Joint Motion Sensing to Make a Determination of a Positional Change of an Individual”) and U.S. Pat. No. 9,700,238 (Stewart, Jul. 11, 2017, “Joint Motion Sensing to Make a Determination of a Positional Change of an Individual”) disclose one or more sensors that produce one or more signals based on one or more joint motions of an individual.

U.S. Pat. No. 8,162,857 (Lanfermann et al., Apr. 24, 2012, “Limb Movement Monitoring System”) and U.S. patent application publication 20100036288 (Lanfermann et al., Feb. 11, 2010, “Limb Movement Monitoring System”) disclose a garment comprising spatially addressable photonic textiles. U.S. patent application publication 20120188158 (Tan et al., Jul. 26, 2012, “Wearable Electromyography-Based Human-Computer Interface”) discloses a plurality of Electromyography (EMG) sensors comprising a human-computer interface (HCl) for interacting with computing systems.

U.S. Pat. No. 8,291,779 (Helmer et al., Oct. 23, 2012, “System and Garment for Detecting Movement”) discloses a system for detecting movement of a limb or section of a limb. U.S. Pat. No. 8,348,865 (Jeong et al., Jan. 8, 2013, “Non-Intrusive Movement Measuring Apparatus and Method Using Wearable Electro-Conductive Fiber”) discloses a non-intrusive movement measuring apparatus and method using wearable electro-conductive fibers. U.S. patent application publication 20130285577 (O'Brien et al., Oct. 31, 2013, “Dielectric Elastomer Self-Sensing Using Plane Approximation”) discloses a method for obtaining feedback parameters related to the state of a dielectric elastomer.

U.S. patent application publications 20140135593 (Jayalth et al., May 15, 2014, “Wearable Architecture and Methods for Performance Monitoring, Analysis, and Feedback”), 20170035354 (Jayalath et al., Feb. 9, 2017, “Wearable Architecture and Methods for Performance Monitoring, Analysis, and Feedback”), and 20200000378 (Jayalath et al., Jan. 2, 2020, “Wearable Architecture and Methods for Performance Monitoring, Analysis, and Feedback”) disclose techniques, fabrics, materials, systems, sensors, EMG sensors, circuitry, algorithms and methods for wearable monitoring devices and associated exercise devices.

U.S. patent application publication 20140240223 (Lake et al., Aug. 28, 2014, “Method and Apparatus for Analyzing Capacitive EMG and IMU Sensor Signals for Gesture Control”) discloses a muscle interface device to control connected devices. U.S. patent U.S. patent application publication 20140240103 (Lake et al., Aug. 28, 2014, “Methods and Devices for Combining Muscle Activity Sensor Signals and Inertial Sensor Signals for Gesture-Based Control”) discloses a wearable electronic EMG device for use with controllable connected devices.

U.S. patent application publication 20150123647 (Gisby et al., May 7, 2015, “Self-Sensing Dielectric Elastomer Device”) discloses circuits, systems and methods for dielectric elastomer device (DED) self-sensing. U.S. Pat. No. 9,043,004 (Casillas et al., May 26, 2015, “Apparel Having Sensor System”), U.S. Pat. No. 9,839,394 (Casillas et al., Dec. 12, 2017, “Apparel Having Sensor System”), U.S. Pat. No. 9,841,330 (Casillas et al., Dec. 12, 2017, “Apparel Having Sensor System”), and U.S. Pat. No. 10,139,293 (Casillas et al., Nov. 27, 2018, “Apparel Having Sensor System”) disclose a plurality of sensors formed of a polymeric material having a conductive particulate material dispersed therein and conductive leads connecting the sensors to a port.

U.S. patent application publications 20150148619 (Berg et al., May 28, 2015, “System and Method for Monitoring Biometric Signals”), 20150230719 (Berg et al., Aug. 20, 2015, “System and Method for Monitoring Biometric Signals”), and 20190261874 (Berg et al., Aug. 29, 2019, “System and Method for Monitoring Biometric Signals”) disclose a garment with a mounting module having an array of connection regions and biometric sensors. U.S. patent application publications 20150148619 (Berg et al., May 28, 2015, “System and Method for Monitoring Biometric Signals”), 20150230719 (Berg et al., Aug. 20, 2015, “System and Method for Monitoring Biometric Signals”), and 20190261874 (Berg et al., Aug. 29, 2019, “System and Method for Monitoring Biometric Signals”) disclose a garment with a mounting module having an array of connection regions and biometric sensors.

U.S. patent application publication 20150305677 (Berg et al., Oct. 29, 2015, “Biometric Electrode System and Method of Manufacture”) discloses an electrode system for EMG sensors with a substrate comprising a reference region and a signal communication region. U.S. patent application publications 20150331533 (McMillen, Nov. 19, 2015, “Flexible Sensors and Applications”) and 20170038881 (McMillen, Feb. 9, 2017, “Flexible Sensors and Applications”) disclose wearable sensors with piezoresistive materials. U.S. patent application publication 20150359455 (Hahami et al., Dec. 17, 2015, “Fiber Optic Shape Sensing Applications”) discloses a fiber optic cable and interrogation circuitry.

U.S. patent application publication 20150359485 (Berg et al., Dec. 17, 2015, “Biometric Signal Conduction System and Method of Manufacture”) discloses EMG sensors with a flexible substrate including a first broad surface and a second broad surface opposing the first broad surface. U.S. patent application publications 20160070347 (McMillen et al., Mar. 10, 2016, “Sensor System Integrated with a Glove”) and 20170303853 (McMillen et al., Oct. 26, 2017, “Sensor System Integrated with a Glove”) disclose sensor systems with piezoresistive material in gloves to measure hand motion.

U.S. Pat. No. 9,322,121 (Dunne et al., Apr. 26, 2016, “Stitched Stretch Sensor”), U.S. Pat. No. 9,885,621 (Dunne et al., Feb. 6, 2018, “Stitched Stretch Sensor”), and U.S. Pat. No. 10,274,384 (Dunne et al., Apr. 30, 2019, “Stitched Stretch Sensor”), and U.S. patent application publication 20140238151 (Dunne et al., Aug. 28, 2014, “Stitched Stretch Sensor”) disclose a stitched sensor including a conductive thread, and the stitch geometry is configured such that an electrical property of the stitched sensor changes based on at least one of stretching, relaxation, or bending of the textile. U.S. patent application publication 20160128632 (Wiebe et al., May 12, 2016, “Garment Integrated Sensing System and Method”) discloses wireless sensor interfaces coupled to a garment, wherein each sensor includes an electrode layer, a positional identifier, and a retention subsystem.

U.S. patent application publications 20160238368 (O'Brien et al., Aug. 18, 2016, “Method of Fabrication of Laminates of Elastic Material Suitable for Dielectric Elastomer Sensing”) and 20180051974 (O'Brien et al., Feb. 22, 2018, “Method of Fabrication of Laminates of Elastic Material Suitable for Dielectric Elastomer Sensing”) disclose a method of fabricating a laminate of flexible and compliant layers of material, such as used to provide a dielectric elastomer sensor. U.S. patent application publications 20160238368 (O'Brien et al., Aug. 18, 2016, “Method of Fabrication of Laminates of Elastic Material Suitable for Dielectric Elastomer Sensing”) and 20180051974 (O'Brien et al., Feb. 22, 2018, “Method of Fabrication of Laminates of Elastic Material Suitable for Dielectric Elastomer Sensing”) disclose a method of fabricating a laminate of flexible and compliant layers of material, such as used to provide a dielectric elastomer sensor.

U.S. patent application publication 20160287175 (Coleman et al., Oct. 6, 2016, “Sensitive, High-Strain, High-Rate, Bodily Motion Sensors Based on Conductive Nano-Material-Rubber Composites”) discloses a process for producing conductive composites. U.S. Pat. No. 9,498,128 (Jayalath et al., Nov. 22, 2016, “Wearable Architecture and Methods for Performance Monitoring, Analysis, and Feedback”) and U.S. Pat. No. 10,413,219 (Jayalath et al., Sep. 17, 2019, “Wearable Architecture and Methods for Performance Monitoring, Analysis, and Feedback”) disclose techniques, fabrics, materials, systems, sensors, EMG sensors, circuitry, algorithms and methods for wearable monitoring devices and associated exercise devices.

U.S. patent application publication 20170036066 (Chahine, Feb. 9, 2017, “Garment with Stretch Sensors”) discloses a knitted or woven garment configured for sensing movement of an adjacent underlying body portion of a wearer of the garment via one or more sensors. U.S. Pat. No. 9,612,102 (Reese et al., Apr. 4, 2017, “Compliant Multi-Region Angular Displacement and Strain Sensors”), and U.S. patent application publications 20160305759 (Reese et al., Oct. 20, 2016, “Compliant Multi-Region Angular Displacement and Strain Sensors”) and 20170168567 (Reese et al., Jun. 15, 2017, “Compliant Multi-Region Angular Displacement and Strain Sensors”) disclose angular displacement sensors and strain sensors multiple motion sensing regions.

U.S. Pat. No. 9,652,101 (McMillen, May 16, 2017, “Two-Dimensional Sensor Arrays”) and U.S. Pat. No. 10,268,315 (McMillen, Apr. 23, 2019, “Two-Dimensional Sensor Arrays”) disclose two-dimensional sensor arrays made with piezoresistive material. U.S. patent application publication 20170171965 (Youn et al., Jun. 15, 2017, “Stretchable Electronic Device and Method of Fabricating the Same”) discloses a stretchable electronic device including a flexible substrate, a conductive fiber pattern formed on the flexible substrate, wherein the conductive fiber pattern has a repetitive circular structure.

U.S. patent application publication 20170191819 (O'Brien et al., Jul. 6, 2017, “Electro-Mechanical Sensor”) discloses an electrical sensor having an electrical capacitance which varies with mechanical deformation. U.S. Pat. No. 9,710,060 (McMillen et al., Jul. 18, 2017, “Sensor System Integrated with a Glove”) and U.S. Pat. No. 10,362,989 (McMillen et al., Jul. 30, 2019, “Sensor System Integrated with a Glove disclose sensor systems with piezoresistive material in gloves to measure hand motion. U.S. Pat. No. 9,753,568 (McMillen, Sep. 5, 2017, “Flexible Sensors and Applications”) and U.S. Pat. No. 10,282,011 (McMillen, May 7, 2019, “Flexible Sensors and Applications”) disclose wearable sensors with piezoresistive materials.

U.S. patent application publication 20170265810 (Van De Vyver, Sep. 21, 2017, “Elastic Sensor”) discloses a stretchable sensor patch comprising an elastic film layer with a stretchability of at least 100% and at least one elastic DEAP strip. U.S. Pat. No. 9,816,799 (Keller et al., Nov. 14, 2017, “Embroidered Strain Sensing Elements”) discloses a deformation sensing fabric comprising a fabric substrate with a first fabric layer and a first conductive element woven into the first fabric layer. U.S. Pat. No. 9,816,800 (O'Brien et al., Nov. 14, 2017, “Method of Fabrication of Laminates of Elastic Material Suitable for Dielectric Elastomer Sensing”) discloses a method of fabricating a laminate of flexible and compliant layers of material, such as used to provide a dielectric elastomer sensor.

U.S. patent application publication 20170347721 (Greenspan et al., Dec. 7, 2017, “Conductive Thread Stitched Stretch Sensor”) discloses conductive thread stitched stretch sensors. U.S. Pat. No. 9,850,600 (Gal, Dec. 26, 2017, “Sensor Garment and Methods of Making the Same”) discloses IP sensor conductors with waveforms having legs that are substantially parallel throughout the operating range of stretch. U.S. Pat. No. 9,874,431 (Reese, Jan. 23, 2018, “Angular Displacement Sensor of Compliant Material”), and U.S. patent application publications 20160033255 (Reese, Feb. 4, 2016, “Angular Displacement Sensor of Compliant Material”) and 20170074637 (Reese, Mar. 16, 2017, “Angular Displacement Sensor of Compliant Material”) disclose multi-directional capacitive sensors. U.S. patent application publication 20180049698 (Berg et al., Feb. 22, 2018, “Garment with Conductive Thread Exposed on Both Sides”) discloses a garment made by bonding an adhesive to a first layer of fabric and a second layer of fabric.

U.S. patent application publications 20180067516 (Longinotti-Buitoni et al., Mar. 8, 2018, “Garments Having Stretchable and Conductive Ink”), 20180376586 (Longinotti-Buitonii et al., Dec. 27, 2018, “Garments Having Stretchable and Conductive Ink”), 20170196513 (Longinotti-Buitoni et al., Jul. 13, 2017, “Garments Having Stretchable and Conductive Ink”), 20150040282 (Longinotti-Buitoni et al., Feb. 12, 2015, “Compression Garments Having Stretchable and Conductive Ink”), and 20140318699 (Longinotti-Buitoni et al., Oct. 30, 2014, “Methods of Making Garments Having Stretchable and Conductive Ink”) disclose garments with stretchable conductive ink patterns.

U.S. Pat. No. 9,913,611 (Wiebe et al., Mar. 13, 2018, “Garment Integrated Sensing System and Method”) and U.S. Pat. No. 10,105,098 (Wiebe et al., Oct. 23, 2018, “Garment Integrated Sensing System and Method”) disclose wireless sensor interfaces coupled to a garment, wherein each sensor includes an electrode layer, a positional identifier, and a retention subsystem. U.S. Pat. No. 9,965,076 (McMillen, May 8, 2018, “Piezoresistive Sensors and Applications”), U.S. Pat. No. 9,546,921 (McMillen et al., Jan. 17, 2017, “Piezoresistive Sensors and Sensor Arrays”), U.S. Pat. No. 10,288,507 (McMillen et al., May 14, 2019, “Piezoresistive Sensors and Sensor Arrays”), and U.S. Pat. No. 9,696,833 (McMillen, Jul. 4, 2017, “Promoting Sensor Isolation and Performance in Flexible Sensor Arrays”) disclose sensors with conductive traces on piezoresistive material with musical applications.

U.S. Pat. No. 10,045,439 (Longinotti-Buitoni et al., Aug. 7, 2018, “Garments Having Stretchable and Conductive Ink”), U.S. Pat. No. 10,258,092 (Longinotti-Buitoni et al., Apr. 16, 2019, “Garments Having Stretchable and Conductive Ink”), U.S. Pat. No. 8,945,328 (Longinotti-Buitoni et al., Feb. 3, 2015, “Methods of Making Garments Having Stretchable and Conductive Ink”), U.S. Pat. No. 8,948,839 (Longinotti-Buitoni et al., Feb. 3, 2015, “Compression Garments Having Stretchable and Conductive Ink”), U.S. Pat. No. 9,817,440 (Longinotti-Buitoni et al., Nov. 14, 2017, “Garments Having Stretchable and Conductive Ink”), and U.S. Pat. No. 9,986,771 (Longinotti-Buitoni et al., Jun. 5, 2018, “Garments Having Stretchable and Conductive Ink”) disclose garments with stretchable conductive ink patterns.

U.S. Pat. No. 10,065,074 (Hoang et al., Sep. 4, 2018, “Training Systems with Wearable Sensors for Providing Users with Feedback”) discloses a training system based on mobile technology and the kinematics of human motion which characterizes, analyzes, and supplies feedback to a user based on the user's movements. U.S. patent application publication 20180279951 (Asnis et al., Oct. 4, 2018, “Movement Compensation for Sensor-Equipped Athletic Garments”) discloses an athletic garment includes connective segments that compensate for motion of an athlete wearing the athletic garment. U.S. Pat. No. 10,119,208 (McMaster, Nov. 6, 2018, “Method for Making Electrically Conductive Textiles and Textile Sensor”) discloses a method for making a textile sensor which includes selecting yarn variables, stitch variables, and/or textile variables.

U.S. Pat. No. 10,143,405 (Jayalath et al., Dec. 4, 2018, “Wearable Performance Monitoring, Analysis, and Feedback Systems and Methods”) and U.S. patent application publication 20140142459 (Jayalath et al., May 22, 2014, “Wearable Performance Monitoring, Analysis, and Feedback Systems and Methods”) disclose techniques, systems, sensors, circuitry, algorithms and methods for wearable monitoring devices. U.S. Pat. No. 10,159,440 (Longinotti-Buitoni et al., Dec. 25, 2018, “Physiological Monitoring Garments”) and U.S. Pat. No. 10,462,898 (Longinotti-Buitoni et al., Oct. 29, 2019, “Physiological Monitoring Garments”) disclose garments for detecting and monitoring physiological parameters such as respiration and cardiac parameters.

U.S. U.S. Pat. No. 10,172,541 (Liao et al., Jan. 8, 2019, “Motion Recognition Device and Method”) and U.S. patent application publication 20170086711 (Liao et al., Mar. 30, 2017, “Motion Recognition Device and Method”) disclose a motion recognition device with a first sense signal portion and a second sense signal portion different from the first sense signal portion. U.S. Pat. No. 10,197,459 (Keller et al., Feb. 5, 2019, “Indexable Strain Sensor”) discloses a deformation sensing apparatus comprising an elastic substrate, a conductive element, and an additional conductive element. U.S. patent application publication 20190046114 (Bogdanovich et al., Feb. 14, 2019, “Garment System Providing Biometric Monitoring”) discloses a garment for monitoring biometric properties of the wearer.

U.S. patent application publication 20190059461 (Walker, Feb. 28, 2019, “Sense-Enabled Apparel”) discloses an apparel piece sized to be worn on a user and a sensor system integrated with the apparel piece. U.S. Pat. No. 10,228,231 (O'Brien et al., Mar. 12, 2019, “Laminated Devices of Elastic Material Suitable for Dielectric Elastomer Sensing”) discloses a laminated device of flexible and compliant layers of material, such as used to provide a dielectric elastomer sensor. U.S. Pat. No. 10,240,265 (McMaster, Mar. 26, 2019, “Method for Optimizing Contact Resistance in Electrically Conductive Textiles”) discloses a method for optimizing contact resistance in electrically conductive yarns and textiles.

U.S. patent application publication 20190117157 (Hu et al., Apr. 25, 2019, “Sensors Facilitating Monitoring of Living Entities”) discloses methods of manufacturing optical strain sensors. U.S. patent application publication 20190132948 (Longinotti-Buitonii et al., May 2, 2019, “Physiological Monitoring Garments”) discloses garments for detecting and monitoring physiological parameters such as respiration and cardiac parameters. U.S. patent application publication 20190145752 (Zhu et al., May 16, 2019, “Highly Stretchable Strain Sensor for Human Motion Monitoring”) discloses a method to assemble a highly stretchable and highly sensitive strain sensor.

U.S. Pat. No. 10,292,652 (Berg et al., May 21, 2019, “System and Method for Monitoring Biometric Signals”) and U.S. Pat. No. 10,321,832 (Berg et al., Jun. 18, 2019, “System and Method for Monitoring Biometric Signals”) disclose a garment with a mounting module having an array of connection regions and biometric sensors. U.S. patent application publication 20190151713 (Berg et al., May 23, 2019, “Printable Electronic Garment Conduit”) discloses an athletic garment with printed EMG sensors. U.S. patent application publication 20190185672 (Boland et al., Jun. 20, 2019, “Viscoelastic Conductive Nanomaterial-Polymer Nanocomposites and Sensing Devices Comprising the Composite Material”) discloses a homogenous composite material with high strength.

U.S. patent application publication 20190228330 (Kaifosh et al., Jul. 25, 2019, “Handstate Reconstruction Based on Multiple Inputs”) discloses methods and systems for recognizing gestures using a plurality of neuromuscular sensors. U.S. Pat. No. 10,378,975 (Sun, Aug. 13, 2019, “Systems, Methods, and Devices for Static and Dynamic Body Measurements”) discloses systems and methods to measure static and dynamic forces of a body using sensors. U.S. patent application publication 20190290198 (Belson et al., Sep. 26, 2019, “Systems and Methods for Monitoring Physical Therapy of the Knee and Other Joints”) discloses systems, devices, and methods for post-surgical joint range of motion measurement, activity monitoring, as well as monitoring compliance.

U.S. Pat. No. 10,362,958 (Morun et al., Jul. 30, 2019, “Systems, Articles, and Methods for Electromyography Sensors”) and U.S. Pat. No. 10,429,928 (Morun et al., Oct. 1, 2019, “Systems, Articles, and Methods for Capacitive Electromyography Sensors”) disclose EMG sensors which coated with protective barriers and adapted to resistively couple to a user's skin. U.S. patent application publication 20190310713 (Wang et al., Oct. 10, 2019, “Sensors for Electronic Finger Devices”) discloses finger-mounted devices with strain sensors and/or ultrasonic sensors to measure finger movement.

U.S. Pat. No. 10,458,866 (Sun, Oct. 29, 2019, “Methods of Manufacturing Devices for Static and Dynamic Body Measurements”) discloses a method of fabricating a sensor for static and dynamic body measurements. U.S. patent application publication 20190342993 (Ahn et al., Nov. 7, 2019, “Stretchable Electronics and Method for Fabricating the Same”) discloses stretchable electronics including a stretchable substrate, support patterns disposed on a surface of the stretchable substrate, and output devices disposed on the patterns. U.S. patent application publication 20190339141 (Gisby et al., Nov. 7, 2019, “A Stretch Sensor with an Improved Flexible Interconnect”) discloses a connection component for a stretchable sensing device.

U.S. Pat. No. 10,488,936 (Baranski et al., Nov. 26, 2019, “Motion and Gesture Input from a Wearable Device”), and U.S. patent application publications 20160091980 (Baranski et al., Mar. 31, 2016, “Motion and Gesture Input from a Wearable Device”) and 20190220099 (Baranski et al., Jul. 18, 2019, “Motion and Gesture Input from a Wearable Device”) disclose wearable devices with optical or EMG sensors that recognize gestures of a user's hand, arm, wrist, and fingers. U.S. patent application publication 20190364983 (Nakajima et al., Dec. 5, 2019, “Wearable Device and Paper Pattern”) discloses a wearable device with sensors, at least one of which is on the front side and the back side respectively.

U.S. Pat. No. 10,502,643 (Keller et al., Dec. 10, 2019, “Resistive-Capacitive Deformation Sensor”) and U.S. Pat. No. 10,067,007 (Keller et al., Sep. 4, 2018, “Resistive-Capacitive Deformation Sensor”) disclose a deformation sensing apparatus which senses stain in two directions. U.S. patent application publication 20190390985 (Kwok et al., Dec. 26, 2019, “Real-Time Surface Shape Sensing for Flexible Structures”) discloses a surface shape sensor in the form of a flexible and stretchable elastomeric substrate with strain/displacement sensing elements embedded in it.

U.S. Pat. No. 10,527,507 (Wood et al., Jan. 7, 2020, “Artificial Skin and Elastic Strain Sensor”) and U.S. Pat. No. 9,797,791 (Vogt et al., Oct. 24, 2017, “Multi-Axis Force Sensing Soft Artificial Skin”), and U.S. patent application publication 20140238153 (Wood et al., Aug. 28, 2014, “Artificial Skin and Elastic Strain Sensor”) disclose an elastic strain sensor with conductive fluid. U.S. patent application publication 20200008715 (Schroeck et al., Jan. 9, 2020, “Rotation Monitoring System and Method”) discloses a rotation monitoring system attached to a limb to identify ranges of motion associated with injuries or poor performance.

U.S. Pat. No. 10,535,278 (Chahine, Jan. 14, 2020, “Garment with Stretch Sensors”) discloses a knitted or woven garment configured for sensing movement of an adjacent underlying body portion of a wearer of the garment via one or more sensors. U.S. patent application publication 20200155069 (Bogdanovich et al., May 21, 2020, “Garment System Providing Biometric Monitoring”) discloses a garment for monitoring a person's respiration, heart function, and motion. U.S. patent application publication 20200237031 (Daniels et al., Jul. 30, 2020, “Fabric, Connections and Functional Structures for Wearable Electronic Garments and Applications for the Same”) discloses a wearable electronic fabric made from interlaced threads.

U.S. patent application publication 20200388192 (Chahine, Dec. 10, 2020, “Garment with Stretch Sensors”) discloses a garment to sense body motion using a plurality of knitted fibers. U.S. patent application publication 20210137418 (Dietz et al., May 13, 2021, “Multibend Shape Sensor”) discloses a multibend sensor with a reference strip and a sliding strip. U.S. patent application publication 20210190556 (Bogdanovich, Jun. 24, 2021, “Super Modular Monitoring System”) discloses a modular monitoring system with a lattice having a plurality of intersections.

U.S. patent application publication 20210190811 (Crockford, Jun. 24, 2021, “System and Method for Monitoring Body Movement”) discloses a method for determining joint flexion or extension using strain gauges and inertial measurement units. U.S. patent application publication 20210255694 (Servati et al., Aug. 19, 2021, “Methods of and Systems for Estimating a Topography of at Least Two Parts of a Body”) discloses a method for estimating body topography by receiving data concerning deformation of the body. U.S. patent application publication 20210307651 (Bogdanovich et al., Oct. 7, 2021, “Objective Range of Motion Monitoring”) discloses a motion monitoring system comprising a deformable wearable device.

U.S. patent application publication 20210393427 (Mirza et al., Dec. 23, 2021, “Wearable System for Evaluating Joint Performance and Function”) discloses a pliable wrap with fluid chambers which are stitched together to conform to a shape of a body joint. U.S. patent application publication 20220008237 (Mirza et al., Jan. 13, 2022, “Wearable System for Evaluating Joint Performance and Function”) discloses a wearable plyowrap with embedded fluid to support a body joint. U.S. patent application publication 20220087565 (Loh et al., Mar. 24, 2022, “Smart Elastic Fabric Tape for Distributed Skin Strain, Movement, and Muscle Engagement Monitoring”) discloses a method of interrogating a sensing mesh using an electrical impedance tomography device.

U.S. patent application publication 20220151557 (Bogdanovich et al., May 19, 2022, “Garment System Providing Biometric Monitoring”) discloses a garment for monitoring a person's respiration, heart function, and motion. U.S. patent application publication 20220205818 (Bogdanovich, Jun. 30, 2022, “Super Modular Monitoring System”) discloses a modular monitoring system with a lattice having a plurality of intersections. U.S. patent application publication 20220214234 (Casillas et al, Jul. 7, 2022, “Apparel Having Sensor System”) discloses a plurality of sensors made from a polymer with conductive particles dispersed therein.

U.S. patent application publication 20230111433 (Dietz et al., Apr. 13, 2023, “Non-Uniform Electrode Spacing with a Bend Sensor”) discloses a multibend sensor with a plurality of electrodes along a sliding strip that are not uniformly distanced. U.S. patent application publication 20230221105 (Ronay et al., Jul. 13, 2023, “Multi-Axis Differential Strain Sensor”) discloses a flexible differential strain sensor, system, and method with a deformable substrate having a first axis and a second axis different than the first axis.

U.S. patent application publication 20230233104 (Caviedes et al., Jul. 27, 2023, “Methods and Systems for Capturing and Visualizing Spinal Motion”) discloses wearable stretch sensors and methods of using them to capture spinal motion and posture information. U.S. patent application publication 20230320625 (Burch et al., Oct. 12, 2023, “Wearable Flexible Sensor Motion Capture System”) discloses a novel system for wearables that captures and stores kinematic data. U.S. patent application publication 20240011851 (Kwok et al., Jan. 11, 2024, “Optical Soft Skin System for Multimodal Sensing”) discloses an optical soft skin system for multimodal sensing comprising a flexible waveguide substrate.

U.S. patent application publication 20240016234 (Samuele et al., Jan. 18, 2024, “Tubular Garment”) discloses methods of manufacturing a tubular garment using a knitting machine. U.S. patent application publication 20240172963 (Carbo et al., May 30, 2024, “Devices, Systems, and Methods to Monitor and Characterize the Motions of a User Via Flexible Circuits”) discloses a tubular body comprising a resilient material, a flexible circuit including a fluid-phase conductor, and an inertial measurement unit. U.S. patent application publication 20240231489 (Kao et al., Jul. 11, 2024, “Wearable Interface Devices with Tactile Functionality”) discloses a wearable tactile interface device which detects environmental inputs.

### SUMMARY OF THE INVENTION

This invention is motion recognition clothing which measures and recognizes changes in body configuration and motion. In an example, motion recognition clothing can measure full-body configuration and motion. Motion recognition clothing includes flexible energy pathways, wherein selected body joints are each spanned by a plurality of flexible energy pathways. Each flexible energy pathway is in energy communication with an energy emitter and an energy receiver. Body joint movements deform the energy pathways. Deformation of the energy pathways changes the transmission of energy from the energy emitters to the energy receivers through the energy pathways. Changes in the transmission of energy are then analyzed to measure and recognize joint movements.

There are many potential human-to-computer interface applications for measurement of full-body configuration and motion, including: directing character actions or entering commands in virtual reality, computer gaming, or telecommunication; gesture recognition as part of a human-computer interface; telerobotics (e.g. remote surgery) or telepresence; animating a virtual character in a movie; guiding dance instruction or recording dance performance motions; and guiding musical instrument instruction or recording instrument playing motions.

There are many potential sports applications for measurement of full-body configuration and motion, including athletic training, motion capture, sports injury prevention, and performance analysis for sports which involve extensive lower-body motion (such as bicycling and soccer) and complex arm and torso motion (such as golf, basketball, baseball, tennis, and Frisbee). There are many potential health, fitness, and medical applications for measurement of full-body configuration and motion, including: medical condition diagnosis (e.g. gait analysis); range of motion assessment; physical therapy; medical therapy (e.g. rehabilitation) evaluation; posture evaluation and correction; posture-related injury prevention; respiratory function assessment; virtual reality exercise; weight management; ambulatory telerobotics; avoidance of repeated motion injuries; and fall prevention and detection.

### BRIEF INTRODUCTION TO THE FIGURES

FIG. 1 shows an upper-body article of clothing (e.g. shirt) and a lower-body article of clothing (e.g. pants) for measuring full-body configuration and motion, wherein major body joints are each spanned by a plurality of flexible energy pathways.

### DETAILED DESCRIPTION OF THE FIGURES

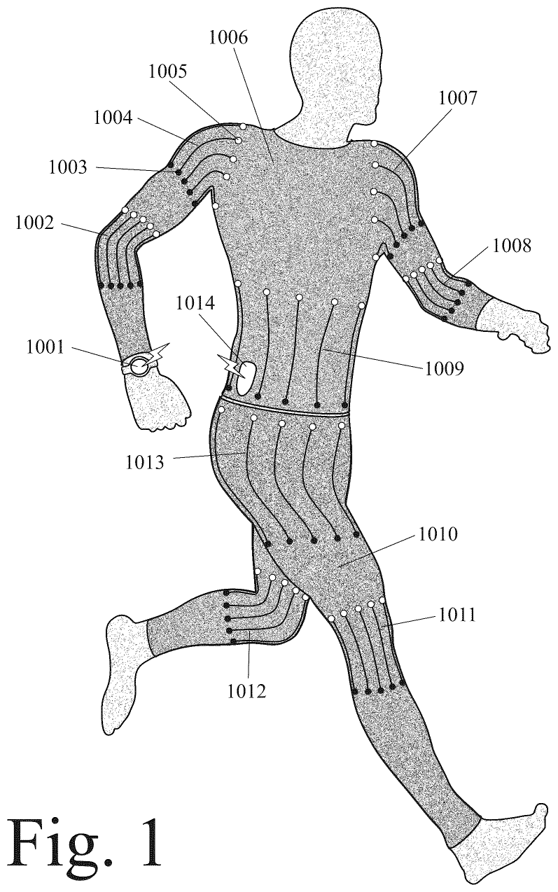

Before discussing the specific embodiment of this invention which is shown in FIG. 1, this disclosure provides an introductory section which covers some of the general concepts, components, and methods which comprise this invention. Where relevant, these concepts, components, and methods can be applied as variations to the example shown in FIG. 1 which is discussed afterwards.

In an example, an upper-body garment or article of clothing for measuring body configuration and motion can comprise: an upper-body garment or article of clothing worn by a person, wherein the garment or article of clothing further comprises; a set of energy pathways which span the person's right elbow; a set of proximally-diverging energy pathways which span the person's right shoulder; a set of energy pathways which span the person's left elbow; a set of proximally-diverging energy pathways which span the person's left shoulder; a set of energy pathways which span a portion of the person's torso or back; a plurality of energy emitters; and a plurality of energy receivers; wherein each energy pathway is in energy communication with an energy emitter which directs energy into the energy pathway and with an energy receiver which measures energy transmission through the energy pathway; and wherein changes in the transmission of energy through energy pathways are analyzed to measure the configuration and motion of body joints.

In an example, a lower-body garment or article of clothing for measuring body configuration and motion can comprise: a lower-body garment or article of clothing worn by a person, wherein the garment or article of clothing further comprises; a set of energy pathways which span the person's right knee; a set of proximally-diverging energy pathways which span the person's right hip; a set of energy pathways which span the person's left knee; a set of proximally-diverging energy pathways which span the person's left hip; a plurality of energy emitters; and a plurality of energy receivers; wherein each energy pathway is in energy communication with an energy emitter which directs energy into the energy pathway and with an energy receiver which measures energy transmission through the energy pathway; and wherein changes in the transmission of energy through energy pathways are analyzed to measure the configuration and motion of body joints.

In an example, electrical and/or electromagnetic energy can be emitted by an energy emitter, transmitted through an energy pathway, and received by an energy receiver. In an example, light energy can be emitted by an energy emitter, transmitted through an energy pathway, and received by an energy receiver. In an example, sonic energy can be emitted by an energy emitter, transmitted through an energy pathway, and received by an energy receiver. In an example, the results of analysis of energy transmission through a first energy pathway can trigger energy transmission through a second energy pathway. In an example, energy transmission through the second energy pathway can require more energy than energy transmission through the first energy pathway.

In an example, an energy pathway can comprise a loop which spans a body joint, wherein ends of the loop are both proximal relative to the joint or are both distal relative to the joint. In an example, an energy pathway can comprise a loop which spans a body joint, wherein an energy emitter in communication with the pathway and an energy receiver in communication with the pathway are both proximal relative to the joint or are both distal relative to the joint. In an example, a first subset of the energy pathways can be substantively parallel to a longitudinal axis of a body joint and a second subset of the energy pathways can be partially-helical around a body member containing the body joint. In an example, a garment or article of clothing can further comprise inertial motion sensors located proximally and distally relative to the body joint, wherein data from the energy pathways and data from the inertial motion sensors are jointly analyzed to measure and/or recognize joint configuration and/or motion.

In an example, a wearable device can measure, recognize, and/or model full-body configuration, posture, and/or motion. In an example, a wearable device can provide mobile, non-intrusive measurement of full-body configuration, posture, and/or motion. In an example, a wearable device can measure full-body kinematics. In an example, a wearable device can measure the angles and/or movements of multiple body joints. In an example, a wearable device can measure the anatomical positions and/or configurations of multiple body joints. In an example, a wearable device can model 3D full-body configuration, posture, and/or motion. In an example, a wearable device can create a 3D model, map, and/or topography of a moving body.

In an example, a wearable device to measure and/or recognize body configuration and/or motion can be a garment (e.g. article of clothing) or clothing accessory. In an example, a wearable device to measure and/or recognize body configuration and/or motion can be incorporated into a garment (e.g. article of clothing) or clothing accessory. In an example, a wearable device to measure and/or recognize body configuration and/or motion can be woven or knitted into a garment (e.g. article of clothing) or clothing accessory. In an example, a wearable device to measure and/or recognize body configuration and/or motion can be attached to a garment (e.g. article of clothing) or clothing accessory. In an example, a wearable device to measure and/or recognize body configuration and/or motion can be sewn or embroidered into a garment (e.g. article of clothing) or clothing accessory. In an example, a wearable device to measure and/or recognize body configuration and/or motion can be printed or adhered onto a garment (e.g. article of clothing) or clothing accessory.

In an example, a wearable device to measure and/or recognize body configuration and/or motion can be integrated (e.g. woven or knitted) into, attached (e.g. sewn, embroidered, or adhered) onto, or printed onto an article of clothing or wearable clothing accessory selected from the group consisting of: full-body suit, jump suit, union suit, dress, leotard, overalls, suit, and uniform; upper body garment, shirt, sweatshirt, t-shirt, undershirt, blouse, hoodie, arm band, arm tube, back brace, bra, cap, coat, collar, elbow brace, elbow pad, elbow tube, finger tube, girdle, glove, hat, headband, jacket, neck band, shoulder pad, shoulder tube, waist band, and waist band; lower body garment, leggings, pants, slacks, sweatpants, tights, trousers, ankle band, ankle tube, boot, hip pad, insole, knee brace, knee pad, knee tube, pantyhose, shoe, shorts, sock, and underpants; and bandage, belt, compression garment, electronic tattoo, joint brace, joint sleeve, patch, strap, underwear, and wearable patch.

In an example, a wearable device to measure and/or recognize body configuration and/or motion can be woven or knitted into an article of clothing. In an example, a wearable device to measure and/or recognize body configuration and/or motion can be sewn or embroidered onto an article of clothing. In an example, a wearable device to measure and/or recognize body configuration and/or motion can be printed or adhered onto an article of clothing. In an example, a wearable device to measure and/or recognize body configuration and/or motion can be inserted into an article of clothing. In an example, a wearable device to measure and/or recognize body configuration and/or motion can be attached to article of clothing by an attachment mechanism selected from the group consisting of: strap, belt, zipper, loop, magnet, hook, melting, clasp, necklace, pin, tape, ring, clip, gluing, adhesion, watch band, bracelet, hook-and-loop textile, buckle, clamp, snap, bangle, weaving, sewing, button, and plug.

In an example, a wearable device to measure and/or recognize body configuration and/or motion can measure movements of some or all of the following body joints: shoulder, elbow, hip, knee, ankle, finger, forearm, jaw, neck, spine, thumb, toe, and wrist. In an example, a wearable device to measure and/or recognize body configuration and/or motion can measure one or more body joint movements selected from the group consisting of: extension, retraction, plantar flexion, bending, inversion, pronation, protraction, radial deviation, rotation, supination, ulnar deviation, flexion, hyperextension, abduction, circumduction, lateral bending, dorsiflexion, eversion, adduction, and elevation.

In an example, a wearable device to measure and/or recognize body configuration and/or motion can measure one or more body joint movements selected from the group consisting of: abduction, adduction, extension, flexion, and/or rotation of the shoulder; eversion, extension, flexion, and/or inversion of the elbow; abduction, adduction, extension, flexion, and/or rotation of the hip; abduction, adduction, extension, and/or flexion of the knee; eversion, extension, flexion, and/or inversion of the ankle; extension and/or flexion of the finger or thumb; extension and/or flexion of the jaw; eversion and/or inversion of the mid-tarsal; abduction, extension, flexion, and/or rotation of the neck; abduction, extension, flexion, lateral bending, and/or rotation of the spine; extension and/or flexion of the toe; and abduction, extension, flexion, and/or ulnar deviation or radial deviation of the wrist.

In an example, a wearable device to measure and/or recognize body configuration and/or motion can comprise: at least one flexible energy (e.g. electrical energy, light energy, sonic energy, or kinetic energy) pathway which spans at least one body joint; at least one energy emitter whose energy is directed into the at least one pathway; and at least one energy receiver which receives energy from the at least one energy emitter after the energy has been transmitted through the at least one pathway; and a data processor, wherein changes in the configuration (e.g. configuration or configurations) of the at least one body joint cause changes in the shape (e.g. shape or shapes) of the at least one pathway, wherein changes in the shape (e.g. shape or shapes) of the at least one pathway cause changes in attributes (e.g. parameters) of the energy transmitted through the at least one pathway, and wherein changes in the attributes (e.g. parameters) of the energy are analyzed in the data processor to measure (e.g. model) changes in the configuration (e.g. configuration or configurations) of the at least one body joint.

In an example, the type of energy which is emitted by an energy emitter, transmitted through an energy pathway, and received by an energy receiver can be electrical and/or electromagnetic energy. In an example, the type of energy which is emitted by an energy emitter, transmitted through an energy pathway, and received by an energy receiver can be light energy. In an example, the type of energy which is emitted by an energy emitter, transmitted through an energy pathway, and received by an energy receiver can be sonic energy. In an example, the type of energy which is emitted by an energy emitter, transmitted through an energy pathway, and received by an energy receiver can be kinetic energy.

In an example, one or more attributes (e.g. parameters) of energy transmitted through an energy pathway can be selected from the group consisting of: the amount (e.g. amount, level, power, amplitude, intensity, or volume) or changes in the amount of energy transmitted through a flexible energy pathway; the current, voltage, resistance, impedance, or capacitance of an energy pathway with respect to energy transmitted through the pathway; and the waveform (e.g. waveform, frequency, phase, or spectral distribution) or changes in the waveform of energy transmitted through a flexible energy pathway.

In an example: motions of one or more body joints change the configurations (e.g. shapes and/or dimensions) of one or more flexible energy pathways which span these body joints; changes in the configurations of these pathways cause changes in one or more parameters of energy transmitted through the energy pathways; and then changes in the one or more parameters are recorded and analyzed to measure and/or recognize body configuration and motion. In an example: motions of one or more body joints deform one or more flexible energy pathways which span these body joints; these deformations cause changes in one or more parameters of energy transmitted through the energy pathways; and then changes in the one or more parameters are recorded and analyzed to measure and/or recognize body configuration and motion. In an example, deformation of a flexible energy pathway can include elongation, expansion, lengthening, stretching, widening, compression, flattening, shrinking, bending, curving, flexing, kinking, rotation, straightening, and/or twisting.

In an example, a wearable device to measure and/or recognize body configuration and/or motion can comprise a plurality of flexible energy pathways which span the same body joint, wherein the pathways have different shapes and/or sizes. In an example, a first energy pathway with a first shape and/or size can be better for measuring a first type of joint motion, a second energy pathway with a second shape and/or size can be better for measuring a second type of joint motion, and combining measurements from both the first and second energy pathways can provide better measurement of a wider range of motion types than either pathway alone. In an example, a first type of energy pathway with a first shape and/or size can be better for measuring a first type of joint motion, a second type of energy pathway with a second shape and/or size can be better for measuring a second type of joint motion, and combining measurements from both types of energy pathways can provide better measurement of a wider variety of motion types than either type of pathway alone.

In an example, a first type of energy pathway can be better for measuring joint motion along a first vector, a second type of energy pathway can be better for measuring joint motion along a second vector, and combining both first and second types of energy pathways can provide more accurate measurement and recognition of overall body configuration and motion than either type alone. In an example, a first type of energy pathway can be better for measuring joint extension, a second type of energy pathway can be better for measuring joint flexion, and combining both first and second types of energy pathways can provide more accurate measurement and recognition of overall body configuration and motion than either type alone. In an example, a first type of energy pathway can be better for measuring joint extension or flexion, a second type of energy pathway can be better for measuring joint torsion or rotation, and combining both first and second types of energy pathways can provide more accurate measurement and recognition of overall body configuration and motion than either type alone.

In an example, a first type of energy pathway can be better for measuring smaller joint motions, a second type of energy pathway can be better for measuring larger joint motions, and combining both first and second types of energy pathways can provide more accurate measurement and recognition of overall body configuration and motion than either type alone. In an example, a first type of energy pathway can be better for measuring slower joint motions, a second type of energy pathway can be better for measuring faster joint motions, and combining both first and second types of energy pathways can provide more accurate measurement and recognition of overall body configuration and motion than either type alone. In an example, a first type of energy pathway can be better for measuring one-time joint motions, a second type of energy pathway can be better for measuring repeated joint motions, and combining both first and second types of energy pathways can provide more accurate measurement and recognition of overall body configuration and motion than either type alone.

In an example, a wearable device to measure and/or recognize body configuration and/or motion can comprise a plurality of flexible energy pathways which span the same body joint, wherein the pathways have different lengths. In an example, a wearable device to measure and/or recognize body configuration and/or motion can comprise a first flexible energy pathway which spans a body joint and a second flexible energy pathway which spans the body joint, wherein the length of the second pathway is at least 25% greater than the length of the first pathway. In an example, there can be a plurality of energy pathways spanning the same body joint, wherein a first subset of these pathways has a first length, wherein a second subset of these pathways has a second length, and wherein the second length is 20% to 50% greater than the first length. In an example, there can be a plurality of energy pathways spanning the same body joint, wherein a first subset of these pathways has a first length, wherein a second subset of these pathways has a second length, and wherein the second length is at least 50% greater than the first length.

In an example, a wearable device to measure and/or recognize body configuration and/or motion can comprise a plurality of flexible energy pathways which span the same body joint, wherein the pathways have different widths. In an example, a wearable device to measure and/or recognize body configuration and/or motion can comprise a first flexible energy pathway which spans a body joint and a second flexible energy pathway which spans the body joint, wherein the width of the second pathway is at least 25% greater than the width of the first pathway. In an example, there can be a plurality of energy pathways spanning the same body joint, wherein a first subset of these pathways has a first width, wherein a second subset of these pathways has a second width, and wherein the second width is 20% to 50% greater than the first width. In an example, there can be a plurality of energy pathways spanning the same body joint, wherein a first subset of these pathways has a first width, wherein a second subset of these pathways has a second width, and wherein the second width is at least 50% greater than the first width.

In an example, a wearable device to measure and/or recognize body configuration and/or motion can comprise a plurality of flexible energy pathways which span the same body joint, wherein the pathways have different thicknesses. In an example, a wearable device to measure and/or recognize body configuration and/or motion can comprise a first flexible energy pathway which spans a body joint and a second flexible energy pathway which spans the body joint, wherein the thickness of the second pathway is at least 25% greater than the thickness of the first pathway. In an example, there can be a plurality of energy pathways spanning the same body joint, wherein a first subset of these pathways has a first thickness, wherein a second subset of these pathways has a second thickness, and wherein the second thickness is 20% to 50% greater than the first thickness. In an example, there can be a plurality of energy pathways spanning the same body joint, wherein a first subset of these pathways has a first thickness, wherein a second subset of these pathways has a second thickness, and wherein the second thickness is at least 50% greater than the first thickness.

In an example, a wearable device to measure and/or recognize body configuration and/or motion can comprise a plurality of flexible energy pathways which span the same body joint, wherein the pathways have different amounts of curvature (e.g. convexity or concavity). In an example, a wearable device to measure and/or recognize body configuration and/or motion can comprise a first flexible energy pathway which spans a body joint and a second flexible energy pathway which spans the body joint, wherein the amount of curvature of the second pathway is at least 25% greater than the amount of curvature of the first pathway. In an example, there can be a plurality of energy pathways spanning the same body joint, wherein a first subset of these pathways has a first amount of curvature, wherein a second subset of these pathways has a second amount of curvature, and wherein the second amount of curvature is 20% to 50% greater than the first amount of curvature. In an example, there can be a plurality of energy pathways spanning the same body joint, wherein a first subset of these pathways has a first amount of curvature, wherein a second subset of these pathways has a second amount of curvature, and wherein the second amount of curvature is at least 50% greater than the first amount of curvature.

In an example, a wearable device to measure and/or recognize body configuration and/or motion can comprise a plurality of flexible energy pathways which span the same body joint, wherein the pathways have different amounts of convolution (e.g. sinusoidal or zigzag undulations). In an example, a wearable device to measure and/or recognize body configuration and/or motion can comprise a first flexible energy pathway which spans a body joint and a second flexible energy pathway which spans the body joint, wherein the amount of convolution (e.g. sinusoidal or zigzag undulations) of the second pathway is at least 25% greater than the amount of convolution of the first pathway. In an example, there can be a plurality of energy pathways spanning the same body joint, wherein a first subset of these pathways has a first amount of convolution (e.g. sinusoidal or zigzag undulations), wherein a second subset of these pathways has a second amount of convolution, and wherein the second amount of convolution is 20% to 50% greater than the first amount of convolution. In an example, there can be a plurality of energy pathways spanning the same body joint, wherein a first subset of these pathways has a first amount of convolution (e.g. sinusoidal or zigzag undulations), wherein a second subset of these pathways has a second amount of convolution, and wherein the second amount of convolution is at least 50% greater than the first amount of convolution.

In an example, a wearable device to measure and/or recognize body configuration and/or motion can comprise a plurality of flexible energy pathways which span the same body joint, wherein the pathways have different undulation frequencies or amplitudes. In an example, a wearable device to measure and/or recognize body configuration and/or motion can comprise a first flexible energy pathway which spans a body joint and a second flexible energy pathway which spans the body joint, wherein the frequency or amplitude of undulations of the second pathway is at least 25% greater than the frequency or amplitude of undulations of the first pathway. In an example, there can be a plurality of energy pathways spanning the same body joint, wherein a first subset of these pathways has a first frequency or amplitude of undulations, wherein a second subset of these pathways has a second frequency or amplitude of undulations, and wherein the second frequency or amplitude of undulations is 20% to 50% greater than the first frequency or amplitude of undulations. In an example, there can be a plurality of energy pathways spanning the same body joint, wherein a first subset of these pathways has a first frequency or amplitude of undulations, wherein a second subset of these pathways has a second frequency or amplitude of undulations, and wherein the second frequency or amplitude of undulations is at least 50% greater than the first frequency or amplitude of undulations.

In an example, a wearable device to measure and/or recognize body configuration and/or motion can comprise a plurality of flexible energy pathways which span the same body joint, wherein different subsets of pathways are staggered (e.g. span different portions) along the longitudinal axis of the body joint (e.g. elbow or knee) or a body member (e.g. arm or leg) which contains the body joint. In an example, a wearable device to measure and/or recognize body configuration and/or motion can comprise a plurality of flexible energy pathways which span the same body joint, wherein different subsets of pathways are longitudinally staggered (e.g. span overlapping but different portions) along the longitudinal axis of the body joint (e.g. elbow or knee) or a body member (e.g. arm or leg) which contains the body joint. In an example, a wearable device to measure and/or recognize body configuration and/or motion can comprise a plurality of flexible energy pathways which span the same body joint, wherein a first subset of these pathways extends further in a proximal direction from the body joint and a second subset of these pathways extends further in a distal direction from the body joint.

In an example, a wearable device to measure and/or recognize body configuration and/or motion can comprise at least flexible energy pathway with a helical, partial-helical, and/or spiral shape. In an example, a wearable device to measure and/or recognize body configuration and/or motion can comprise at least flexible energy pathway with a helical (or partial-helical) shape which encircles (or partially-encircles) a body member (e.g. arm or leg) which contains a body joint. In an example, a wearable device to measure and/or recognize body configuration and/or motion can comprise at least flexible energy pathway with a helical (or partial-helical) shape which spans and encircles (or partially-encircles) a body joint.

In an example, a helical energy pathway can encircle (in a helical manner) a body member containing a body joint at least once, wherein encircling in a helical manner means that pathway spans a portion of the length of the body member as well as spanning the member circumferentially. In an example, a helical energy pathway can encircle (in a helical manner) a body member containing a body joint at least twice, wherein encircling in a helical manner means that pathway spans a portion of the length of the body member as well as spanning the member circumferentially. In an example, a partially-helical energy pathway can partially-encircle (in a helical manner) a body member containing a body joint, spanning between 50% and 90% of the circumference of the body member, wherein encircling in a helical manner means that pathway spans a portion of the length of the body member as well as spanning the member circumferentially. In an example, a wearable device to measure and/or recognize body configuration and/or motion can comprise a first flexible energy pathway which spans a body joint in a longitudinal manner and a second flexible energy pathway which spans the body joint in a helical or partially-helical manner.

In an example, a flexible energy pathway can have an undulating shape. In an example, a flexible energy pathway can have a sinusoidal shape. In an example, a flexible energy pathway can have a serpentine shape. In an example, a flexible energy pathway can have a saw-tooth shape. In an example, a flexible energy pathway can have a zigzag shape. In an example, a flexible energy pathway can have a horseshoe shape. In an example, a flexible energy pathway can have a meandering shape. In an example, a flexible energy pathway can have an oscillating shape. In an example, a flexible energy pathway can have a square-wave shape. In an example, a flexible energy pathway can have a tessellating shape. In an example, a flexible energy pathway can have longitudinal undulations (e.g. undulations around a central longitudinal axis). In an example, a flexible energy pathway can have cross-sectional undulations (e.g. undulations in width or thickness).

In an example, there can be a plurality of undulating (e.g. sinusoidal, serpentine, and/or zigzag) energy pathways spanning the same body joint, wherein a first subset of these pathways has a first undulation amplitude, wherein a second subset of these pathways has a second undulation amplitude, and wherein the second undulation amplitude is 20% to 50% greater than the first undulation amplitude. In an example, there can be a plurality of undulating (e.g. sinusoidal, serpentine, and/or zigzag) energy pathways spanning the same body joint, wherein a first subset of these pathways has a first undulation amplitude, wherein a second subset of these pathways has a second undulation amplitude, and wherein the second undulation amplitude is at least 50% greater than the first undulation amplitude.

In an example, there can be a plurality of undulating (e.g. sinusoidal, serpentine, and/or zigzag) energy pathways spanning the same body joint, wherein a first subset of these pathways has a first undulation frequency, wherein a second subset of these pathways has a second undulation frequency, and wherein the second undulation frequency is 20% to 50% greater than the first undulation frequency. In an example, there can be a plurality of undulating (e.g. sinusoidal, serpentine, and/or zigzag) energy pathways spanning the same body joint, wherein a first subset of these pathways has a first undulation frequency, wherein a second subset of these pathways has a second undulation frequency, and wherein the second undulation frequency is at least 50% greater than the first undulation frequency.

In an example, a flexible energy pathway can comprise a loop. In an example, a flexible energy pathway can comprise a loop which spans a body joint. In an example, a flexible energy pathway can comprise one or more loops. In an example, a flexible energy pathway can comprise two loops. In an example, a flexible energy pathway can comprise a loop which spans a body joint, wherein both ends of the loop and/or pathway are proximal relative to the joint. In an example, a flexible energy pathway can comprise a loop which spans a body joint, wherein both ends of the loop and/or pathway are distal relative to the joint. In an example, a flexible energy pathway can comprise a loop which spans a body joint, wherein both an energy emitter and an energy receiver which are in energy communication with the pathway are proximal relative to the joint. In an example, a flexible energy pathway can comprise a loop which spans a body joint, wherein both an energy emitter and an energy receiver which are in energy communication with the pathway are distal relative to the joint.

In an example, a first segment of an energy pathway can have a first cross-sectional shape and a second segment of the energy pathway can have a second cross-sectional shape. In an example, a first segment of an energy pathway can have a first cross-sectional size and a second segment of the energy pathway can have a second cross-sectional size. In an example, an energy pathway can be longitudinally tapered, wherein a proximal portion of the pathway has a smaller cross-sectional size than a distal portion of the pathway. In an example, an energy pathway can be longitudinally tapered, wherein a proximal portion of the pathway has a larger cross-sectional size than a distal portion of the pathway. In an example, an energy pathway can comprise a longitudinal series of tapered segments.

In an example, there can be longitudinal variation in the cross-sectional size of an energy pathway. In an example, there can be longitudinal variation in the cross-sectional shape of an energy pathway. In an example, there can be longitudinal variation in the cross-sectional shape of an energy pathway along the length of the pathway. In an example, there can be longitudinal variation in the cross-sectional size of an energy pathway along the length of the pathway. In an example, there can be longitudinal variation in the curvature (e.g. convexity or concavity) of an energy pathway. In an example, there can be longitudinal variation in the material comprising an energy pathway. In an example, an energy pathway can have an asymmetric core around a longitudinal axis. In an example, an energy pathway can have cross-sectional and/or core eccentricity. In an example, the core of an energy pathway can be asymmetric and/or eccentric.

In an example, there can be longitudinal variation in the cross-sectional size of a flexible energy pathway. In an example, there can be longitudinal variation in the cross-sectional shape of a flexible energy pathway. In an example, there can be longitudinal variation in the cross-sectional structure of a flexible energy pathway. In an example, there can be longitudinal variation in the material comprising a flexible energy pathway. In an example, there can be longitudinal variation in the cross-sectional (e.g. core) symmetry or asymmetry of a flexible energy pathway. In an example, there can be longitudinal variation in the cross-sectional (e.g. core) eccentricity of a flexible energy pathway. In an example, there can be longitudinal variation in the number of bundled sub-pathways in a flexible energy pathway. In an example, there can be longitudinal variation in the cross-sectional configuration of bundled sub-pathways in a flexible energy pathway.

In an example, there can be longitudinal variation in the cross-sectional size of an energy pathway. In an example, the cross-sectional size an energy pathway can increase in a proximal-to-distal direction. In an example, the cross-sectional size an energy pathway can decrease in a proximal-to-distal direction. In an example, the cross-sectional size of the proximal end of an energy pathway can be greater than the cross-sectional size of the distal end of the energy pathway. In an example, the cross-sectional size of the proximal end of an energy pathway can be less than the cross-sectional size of the distal end of the energy pathway.

In an example, there can be longitudinal variation in the cross-sectional shape of an energy pathway. In an example, the cross-sectional shape of a proximal portion of an energy pathway can be more convex than the cross-sectional shape of a distal portion of the energy pathway. In an example, the cross-sectional shape of a proximal portion of an energy pathway can be more arcuate than the cross-sectional shape of a distal portion of the energy pathway. In an example, the cross-sectional shape of a proximal portion of an energy pathway can be less convex than the cross-sectional shape of a distal portion of the energy pathway. In an example, the cross-sectional shape of a proximal portion of an energy pathway can be less arcuate than the cross-sectional shape of a distal portion of the energy pathway.

In an example, there can be longitudinal variation in the amount of undulation (e.g. the frequency or amplitude of undulations) in an energy pathway. In an example, the proximal portion of an energy pathway can be more undulating (e.g. have more or larger undulations) than a distal portion of the energy pathway. In an example, the proximal portion of an energy pathway can be less undulating (e.g. have fewer or smaller undulations) than a distal portion of the energy pathway. In an example, there can be longitudinal variation in the shape (e.g. the waveform) of undulations in an energy pathway.

In an example, a plurality of energy pathways which span the same body joint can collectively form a 3D mesh, grid, or lattice with quadrilateral openings. In an example, two energy pathways which span the same body joint can have different diameters. In an example, a wearable device can include a plurality of energy pathways spanning the same body joint, wherein a subset of these pathways are substantively straight when the body joint is in an extended configuration and wherein a subset of these pathways are arcuate when the body joint is in the extended configuration.

In an example, a wearable device can include a plurality of energy pathways spanning the same body joint, wherein a subset of these pathways are substantively parallel to a longitudinal axis of the body joint, and wherein a subset of these pathways are partially-helical (e.g. spanning less than one complete rotation around a body member). In an example, a plurality of energy pathways which span the same body joint can collectively form a 3D mesh, grid, or lattice with square, rhomboid, or trapezoid shaped openings. In an example, two energy pathways which span the same body joint can have different lengths.

In an example, a plurality of energy pathways can span the same body joint in a substantially-parallel manner relative to a longitudinal axis of the joint. In an example, four energy pathways can span the same body joint on four radial quadrants, respectively, of the joint. In an example, a plurality of energy pathways which span the same body joint can be nested and/or concentric. In an example, two energy pathways which span the same body joint can be knitted together in loops or chains. In an example, a plurality of energy pathways which span the same body joint can be separated by a constant distance.

In an example, two energy pathways which span the same body joint can be linked together. In an example, a plurality of energy pathways which span the same body joint can be separated by a constant number of radial degrees. In an example, a plurality of energy pathways can span the same body joint, wherein at least one pathway is tangential to another pathway. In an example, two energy pathways which span the same body joint (or virtual extensions of them in 3D space) can be substantially parallel to each other.

In an example, a plurality of energy pathways which span the same body joint can collectively form a mesh, grid, or lattice with a plurality of diamond-shaped, rectangular, parallelogram-shaped, or other polygonal openings. In an example, a plurality of energy pathways which span the same body joint can collectively form a mesh, grid, or lattice with a tessellating geometric pattern. In an example, a plurality of overlapping and/or intersecting energy pathways can span the same body joint. In an example, a wearable device can include a plurality of energy pathways which span the same body joint and are bundled together.

In an example, a wearable device can include a plurality of energy pathways which span the same body joint and are woven, braided, or plaited together. In an example, a plurality of energy pathways which span the same body joint can collectively form a 3D mesh, grid, or lattice. In an example, two energy pathways which span the same body joint can be braided, plaited, and/or interwoven. In an example, a plurality of energy pathways which span the same body joint can collectively form a 3D mesh, grid, or lattice with hexagonal openings. In an example, two energy pathways which span the same body joint (or virtual extensions of them in 3D space) can be substantially diagonal and/or oblique relative to each other.

In an example, a plurality of energy pathways can span the same body joint in a substantially-parallel manner. In an example, four energy pathways can span the same body joint at different radial locations around the circumference of the joint (e.g. the body member containing the joint). In an example, a plurality of energy pathways can span the same body joint in an oblique, diagonal, and/or traverse manner. In an example, four energy pathways can span the same body joint on four radial quadrants of the joint (e.g. ventral quadrant, dorsal quadrant, and two side quadrants between the ventral and dorsal quadrants).

In an example, a plurality of energy pathways which span the same body joint can collectively form a 3D mesh, grid, or lattice with triangular openings. In an example, two energy pathways which span the same body joint can be bundled together. In an example, a plurality of energy pathways can span the same body joint in an oblique, diagonal, and/or traverse manner relative to each other. In an example, two energy pathways which span the same body joint (or virtual extensions of them in 3D space) can intersect at an acute angle.

In an example, a plurality of energy pathways can span the same body joint at different radial locations around the circumference of the joint (e.g. the body member containing the joint). In an example, two energy pathways which span the same body joint (or virtual extensions of them in 3D space) can be perpendicular and/or orthogonal to each other. In an example, a wearable device can include a plurality of energy pathways spanning the same body joint, wherein a subset of these pathways are substantively parallel to a longitudinal axis of the body joint, and wherein a subset of these pathways are not parallel with (e.g. are oblique with respect to) the longitudinal axis of the body joint.

In an example, a plurality of energy pathways can span the same body joint in a substantially-parallel manner relative to each other. In an example, four energy pathways can span the same body joint at different radial locations around the circumference of the joint (e.g. the body member containing the joint), including a ventral location, a dorsal location, and two side locations between the ventral and dorsal locations. In an example, a wearable device can include a plurality of energy pathways spanning the same body joint, wherein a subset of these pathways are substantively parallel to a longitudinal axis of the body joint, and wherein a subset of these pathways are helical.

In an example, a plurality of energy pathways can span the same body joint in an oblique, diagonal, and/or traverse manner relative to a longitudinal axis of the joint. In an example, two energy pathways which span the same body joint (or virtual extensions of them in 3D space) can intersect at a right angle. In an example, a wearable device can comprise four energy pathways: a first pathway which spans the ventral surface of the joint, a second pathway which spans the dorsal surface of the joint, and third and fourth pathways which span opposing-side lateral surfaces of the joint.

In an example, a wearable device can comprise a plurality of longitudinal energy pathways spanning the same body joint, wherein proximal ends of the energy pathways are separated by a first average distance, wherein distal ends of the pathways are separated by a second average distance, wherein the second average distance is less than the first average distance, and wherein proximal means closer to a person's heart when the person is in the Vitruvian Man body configuration. In an example, a wearable device can comprise a plurality of longitudinal energy pathways spanning the same body joint, wherein proximal ends of the energy pathways are a first average distance apart, wherein distal ends of the pathways are a second distance apart, wherein the second distance is less than the first distance, and wherein proximal means closer to a person's heart when the person is in the Vitruvian Man body configuration.

In an example, a wearable device can comprise a plurality of longitudinal energy pathways spanning the same body joint, wherein proximal ends of the energy pathways are separated by a first average distance, wherein distal ends of the pathways are separated by a second average distance, wherein the second average distance is at least 10% less than the first average distance, and wherein proximal means closer to a person's heart when the person is in the Vitruvian Man body configuration. In an example, a wearable device can comprise a plurality of longitudinal energy pathways spanning the same body joint, wherein proximal ends of the energy pathways are a first average distance apart, wherein distal ends of the pathways are a second distance apart, wherein the second distance is at least 10% less than the first distance, and wherein proximal means closer to a person's heart when the person is in the Vitruvian Man body configuration.

In an example, a plurality of energy pathways spanning the same body joint can be proximally-converging, meaning that their proximal ends are closer together than their distal ends, wherein proximal means closer to a person's heart when the person is in the Vitruvian Man body configuration. In an example, a plurality of energy pathways spanning the same body joint can collectively comprise a tapered 3D shape, wherein proximal ends of the pathways are farther apart than distal ends of the pathways. In an example, a plurality of energy pathways spanning the same body joint can have a common point of convergence. In an example, a plurality of energy pathways spanning the same body joint can partially converge in a distal direction.

In an example, a plurality of energy pathways spanning the same body joint can partially converge in a proximal direction. In an example, a plurality of energy pathways spanning the same body joint can collectively comprise a tapered 3D shape, wherein proximal ends of the pathways are closer together than distal ends of the pathways. In an example, a plurality of energy pathways spanning the same body joint can be proximally-diverging, meaning that their proximal ends are farther apart than their distal ends, wherein proximal means closer to a person's heart when the person is in the Vitruvian Man body configuration. In an example, a plurality of energy pathways spanning the same body joint can collectively comprise a tapered 3D shape.

In an example, a first energy pathway can span the dorsal surface of a body member containing a body joint, a second energy pathway can span the ventral surface of that body member, a third energy pathway can span a first lateral surface of that body member, and a fourth energy pathway can span a second lateral surface of that body member. In an example, the motion and/or configuration of a body joint can be measured using multiple energy pathways which span a portion of the body member which contains the body joint, wherein these energy pathways span longitudinally-sequential cross-sectional perimeters of the body member along radial angles, latitudes, or polar coordinates which are evenly distributed around the 0 to 360 degree range.

In an example, a plurality of energy pathways can span longitudinally-sequential cross-sectional perimeters of a body member containing a body joint along radial angles, latitudes, or polar coordinates of approximately 0, 45, 90, 135, 180, 225, 270, and 315 degrees. In an example, a plurality of energy pathways can span a portion of a body member containing a body joint which contains the body joint, wherein these energy pathways span longitudinally-sequential cross-sectional perimeters of a body member containing a body joint along radial angles, latitudes, or polar coordinates of approximately 0, 60, 120, 180, 240, and 300 degrees. In an example, a plurality of energy pathways can span a portion of a body member containing a body joint which contains the body joint, wherein these energy pathways span longitudinally-sequential cross-sectional perimeters of a body member containing a body joint along radial angles, latitudes, or polar coordinates of approximately 0, 90, 180, and 270 degrees.

In an example, a flexible energy pathway can be incorporated into an article of clothing or clothing accessory by weaving or knitting. In an example, a wearable device to measure and/or recognize body configuration and/or motion can include an energy pathway comprising a plain weave, rib weave, basket weave, twill weave, satin weave, or leno weave. In an example, a wearable device to measure and/or recognize body configuration and/or motion can include an energy pathway comprising with bendable fibers, threads, or yarns. In an example, a wearable device to measure and/or recognize body configuration and/or motion can include an energy pathway comprising a bendable layer, trace, or substrate.

In an example, a wearable device to measure and/or recognize body configuration and/or motion can include an energy pathway comprising elastic fibers, threads, or yarns. In an example, a wearable device to measure and/or recognize body configuration and/or motion can include an energy pathway comprising an elastic layer, trace, or substrate. In an example, a wearable device to measure and/or recognize body configuration and/or motion can include an energy pathway comprising electroconductive fibers, threads, or yarns. In an example, a flexible energy pathway can be woven or knit into fabric which is used to make an article of clothing or clothing accessory.

In an example, a wearable device to measure and/or recognize body configuration and/or motion can include an energy pathway comprising an electronically-functional bandage. In an example, a wearable device to measure and/or recognize body configuration and/or motion can include an energy pathway comprising an electronically-functional tattoo. In an example, a wearable device to measure and/or recognize body configuration and/or motion can include an energy pathway comprising interlaced fibers, threads, or yarns.

In an example, a wearable device to measure and/or recognize body configuration and/or motion can include an energy pathway comprising sinusoidal or zigzagging fibers, threads, or yarns. In an example, a wearable device to measure and/or recognize body configuration and/or motion can include an energy pathway comprising stretchable fibers, threads, or yarns. In an example, a wearable device to measure and/or recognize body configuration and/or motion can include an energy pathway comprising stretchable layer, trace, or substrate. In an example, a wearable device to measure and/or recognize body configuration and/or motion can include an energy pathway comprising a layer or coating of metallic nanoparticles.

In an example, a wearable device to measure and/or recognize body configuration and/or motion can include an energy pathway comprising a graphene layer. In an example, a plurality of energy pathways can span the same body joint, wherein two or more of the pathways differ in their flexibility and/or elasticity. In an example, an energy pathway can comprise multiple parallel layers. In an example, a plurality of energy pathways can span the same body joint, wherein two or more of the pathways differ in their transparency.

In an example, a first portion (e.g. half) of an energy pathway can have a first level of undulation or convolution and a second portion (e.g. half) of an energy pathway can have a second level of undulation or convolution, wherein the second level is greater than the first level. In an example, a plurality of energy pathways can span the same body joint, but have convexities or concavities which face in different directions. In an example, a plurality of energy pathways can span the same body joint, but have different degrees of curvature (e.g. convexity or concavity).

In an example, a plurality of energy pathways can span the same body joint, but have different amounts of undulation and/or convolution. In an example, a plurality of energy pathways can span the same body joint, wherein two or more of the pathways differ in the directions of their convexities or concavities. In an example, a plurality of energy pathways can span the same body joint, but have different levels of flexibility and/or elasticity. In an example, a plurality of energy pathways can span the same body joint, but have different levels of resistance and/or impedance.

In an example, a plurality of energy pathways can span the same body joint, but have different levels of transparency. In an example, a plurality of energy pathways can span the same body joint, wherein two or more of the pathways differ in their degrees of curvature (e.g. convexity or concavity). In an example, a plurality of energy pathways can span the same body joint, wherein two or more of the pathways differ in their amounts of undulation and/or convolution.

In an example, a first portion (e.g. half) of an energy pathway can have a convexity or concavity which faces in a first direction and a second portion (e.g. half) of an energy pathway can have a convexity or concavity which faces in a second direction. In an example, a first portion (e.g. half) of an energy pathway can have a first level of flexibility and/or elasticity and a second portion (e.g. half) of an energy pathway can have a second level of flexibility and/or elasticity, wherein the second level is greater than the first level.

In an example, a plurality of energy pathways can span the same body joint, wherein two or more of the pathways differ in their resistance and/or impedance levels. In an example, a first portion (e.g. half) of an energy pathway can have a first level of resistance and/or impedance and a second portion (e.g. half) of an energy pathway can have a second level of resistance and/or impedance, wherein the second level is greater than the first level.

In an example, a plurality of energy pathways can span the same body joint, wherein two or more of the pathways differ in the angles at which they span the joint. In an example, an energy pathway can comprise two energy-conducting layers which are separated by a layer which does not conduct energy. In an example, a first portion (e.g. half) of an energy pathway can have first degree of curvature and a second portion (e.g. half) of an energy pathway can have a second degree of curvature, wherein the second degree is greater than the first degree.

In an example, an energy pathway can comprise multiple layers. In an example, a first portion (e.g. half) of an energy pathway can have a first level of transparency and a second portion (e.g. half) of an energy pathway can have a second level of transparency, wherein the second level is greater than the first level. In an example, an energy pathway can comprise multiple layers, wherein some of the layer conduct energy and some do not.

In an example, two energy pathways spanning the same body joint can differ in deformability. In an example, proximal and distal sections of an energy pathway can differ in malleability. In an example, two longitudinal sections of an energy pathway can differ in bendability. In an example, two longitudinal sections of an energy pathway can differ in flexibility. In an example, two energy pathways spanning the same body joint can differ in flexibility. In an example, a wearable energy pathway for measuring joint movement can be flexible.

In an example, proximal and distal sections of an energy pathway can differ in deformability. In an example, two energy pathways spanning the same body joint can differ in stretchability. In an example, two energy pathways spanning the same body joint can differ in bendability. In an example, two energy pathways spanning the same body joint can differ in elasticity. In an example, a wearable energy pathway for measuring joint movement can be compressible. In an example, proximal and distal sections of an energy pathway can differ in elasticity.

In an example, two energy pathways spanning the same body joint can differ in compressibility. In an example, two energy pathways spanning the same body joint can differ in pliability. In an example, two energy pathways spanning the same body joint can malleability. In an example, two longitudinal sections of an energy pathway can differ in compressibility. In an example, two longitudinal sections of an energy pathway can differ in elasticity.

In an example, a wearable energy pathway for measuring joint movement can be pliable. In an example, proximal and distal sections of an energy pathway can differ in compressibility. In an example, a wearable energy pathway for measuring joint movement can malleable. In an example, proximal and distal sections of an energy pathway can differ in pliability. In an example, a wearable energy pathway for measuring joint movement can be deformable. In an example, two longitudinal sections of an energy pathway can differ in pliability. In an example, a wearable energy pathway for measuring joint movement can be stretchable. In an example, proximal and distal sections of an energy pathway can differ in softness.

In an example, two energy pathways spanning the same body joint can differ in softness. In an example, a wearable energy pathway for measuring joint movement can be bendable. In an example, proximal and distal sections of an energy pathway can differ in flexibility. In an example, a wearable energy pathway for measuring joint movement can be twistable. In an example, proximal and distal sections of an energy pathway can differ in bendability.

In an example, two longitudinal sections of an energy pathway can differ in stretchability. In an example, a wearable energy pathway for measuring joint movement can be soft. In an example, two longitudinal sections of an energy pathway can differ in malleability. In an example, two longitudinal sections of an energy pathway can differ in softness. In an example, a wearable energy pathway for measuring joint movement can be elastic. In an example, proximal and distal sections of an energy pathway can differ in stretchability. In an example, two longitudinal sections of an energy pathway can differ in deformability.

In an example, there can be differences in the types and/or levels of (electrical, optical, or sonic) energy which are transmitted through different energy pathways which span the same body joint. Having different energy pathways transmit different types and/or levels of energy can provide more accurate measurement of the joint's motion than having all energy pathways transmit the same type and/or level of energy. In an example, there can be differences in the waveforms or frequencies of energy transmitted through different energy pathways which span the same body joint. In an example, a first energy pathway spanning a body joint can transmit a first level of (electrical, optical, or sonic) energy and a second energy pathway spanning the body joint can transmit a second level of (electrical, optical, or sonic) energy.

In an example, an energy pathway can span a single body joint. In an example, an energy pathway can span multiple (e.g. a longitudinal series of) body joints. In an example, a first energy pathway can span a first body joint and a second energy pathway can span both the first body joint and a second body joint. In an example, the first energy pathway and the second energy pathway be substantially parallel to each other as they span the first body joint. In an example, a plurality of energy pathways can span a plurality of body joints in order to measure full-body configuration and motion, wherein a subset of this plurality of energy pathways each span a single body joint and a subset of this plurality of energy pathways each span multiple (e.g. a longitudinal series of) body joints.

In an example, an energy pathway can comprise a radially-symmetric bundle of sub-pathways (e.g. energy-conducting fibers or strips). In an example, an energy pathway can comprise a bundle of energy-conducting fibers. In an example, an energy pathway can comprise a woven or braided bundle of sub-pathways (e.g. energy-conducting fibers or strips). In an example, the core of an energy pathway can comprise a bundle of energy-conducting fibers. In an example, an energy pathway can comprise a bundle of sub-pathways (e.g. energy-conducting fibers or strips), wherein deformation of the energy pathway affects the sub-pathways in different (e.g. asymmetric) ways.

In an example, an energy pathway can comprise a bundle of sub-pathways (e.g. energy-conducting fibers or strips), wherein deformation (e.g. bending, stretching, elongation, or compression) of the energy pathway in a first direction selectively reduces energy transmission through a first sub-set of sub-pathways and wherein deformation (e.g. e.g. bending, stretching, elongation, or compression) of the energy pathway in a second different selectively reduces energy transmission through a second sub-set of sub-pathways.

In an example, an energy pathway can comprise a multi-channel pathway with a plurality of energy-conducting fibers. In an example, a cross-section of an energy pathway can comprise between three and six sub-pathways (e.g. energy-conducting fibers or strips). In an example, an energy pathway can comprise a bundle of three to six energy-conducting fibers. In an example, an energy pathway can comprise a radially-hexagonal bundle of six sub-pathways (e.g. energy-conducting fibers or strips). In an example, an energy pathway can comprise a bundle of four to six sub-pathways (e.g. energy-conducting fibers or strips).

In an example, an energy pathway can comprise a bundle of sub-pathways (e.g. energy-conducting fibers or strips). In an example, a cross-section of an energy pathway can comprise between three and six sub-pathways (e.g. energy-conducting fibers or strips) with arcuate (e.g. circular) cross-sections. In an example, an energy pathway can comprise a radially-asymmetric bundle of sub-pathways (e.g. energy-conducting fibers or strips). In an example, an energy pathway can comprise a bundle of six to eight sub-pathways (e.g. energy-conducting fibers or strips).

In an example, an energy pathway can span a body joint (e.g. the body member containing the joint) in a helical manner, wherein spanning in a helical manner means that the pathway curves in both longitudinal and circumferential directions as it spans the joint. In an example, a helical energy pathway can help to measure torsion and/or rotation of a body joint or body member. In an example, a first energy pathway can span a body joint in a longitudinal manner (e.g. only in a longitudinal direction) and a second energy pathway can span the same body joint in a helical manner (e.g. in both longitudinal and circumferential directions). In an example, a helical energy pathway can span the entire circumference of a body joint (e.g. the body member containing the joint) between one and three times. In an example, a partially-helical energy pathway can span between 50% and 90% of the circumference of a body joint.

In an example, an energy pathway can be substantially straight when a body joint which is spans is in an extended configuration. In an example, an energy pathway can have lateral waves (e.g. lateral undulations, sinusoidal waves, zigzag waves, or square waves) along its longitudinal axis. In an example, an energy pathway can have lateral waves (e.g. lateral undulations, sinusoidal waves, zigzag or sawtooth waves, or square waves) along its longitudinal axis, wherein the distance between waves is increased when the energy pathway is elongated and/or stretched. In an example, an energy pathway can have a tessellating (e.g. geometrically-repeating) pattern its longitudinal axis. In an example, a plurality of energy pathways spanning a body joint can be stacked, wherein a first energy pathway is a first distance from the surface of a person's body and a second pathway is a second distance from this surface.

In an example, an energy pathway can be made by doping, impregnating, and/or coating an elastomeric polymer (e.g. PDMS) with energy-conductive metal (e.g. silver or aluminum) or carbon structures (e.g. carbon nanotubes). In an example, an energy pathway can be made with an elastomeric polymer such as polydimethylsiloxane (PDMS), ethylene propylene dieneterpolymer (EPDM), polyaniline, polyethylene terephthalate (PET), polyurethane laminated fabric (PUL), thermoplastic polyurethane (TPU), and/or electroactive gel. In an example, an energy pathway can be made with aluminum, carbon nanotubes, gold, graphene, nickel, silver, and/or steel.

In an example, an energy pathway can be made by adhesion and/or gluing. In an example, an energy pathway can be made by knitting or weaving with energy-conductive thread or yarn. In an example, an energy pathway can be made with cotton, denim, elastane, linen, nylon, polyester, rayon, silk, spandex, and/or wool. In an example, an energy pathway can be made by printing with an energy-conductive link. In an example, an energy pathway can be made with copper or copper alloy. In an example, an energy pathway can be made with a transparent elastomeric polymer material. In an example, an energy pathway can be made by doping, impregnating, and/or coating an elastomeric polymer material with energy-conductive metal particles or carbon structures. In an example, an energy pathway can be made by sewing and/or embroidering with energy-conductive thread or yarn.

In an example, an energy pathway can comprise an energy-conducting mesh, grid, or lattice which spans a body joint, wherein spaces and/or elements in the mesh, grid, or lattice have triangular shapes. In an example, an energy pathway can comprise an energy-conducting mesh, grid, or lattice which spans a body joint, wherein one or more modular energy emitters can be reversibly attached to different locations on the mesh, grid, or lattice. In an example, a plurality of energy pathways which span a body joint can collectively comprise an orthogonal energy-conducting mesh, grid, or lattice.

In an example, an energy pathway can comprise an energy-conducting mesh, grid, or lattice which spans a body joint, wherein a plurality of energy emitters in energy communication with the mesh, grid, or lattice are activated at different times to measure energy transmissions in different directions and/or over different distances. In an example, an energy pathway can comprise an energy-conducting mesh, grid, or lattice which spans a body joint, wherein one or more modular energy receivers can be reversibly attached to different locations on the mesh, grid, or lattice. In an example, an energy pathway can comprise an orthogonal energy-conducting mesh, grid, or lattice.

In an example, an energy pathway can comprise an energy-conducting mesh, grid, or lattice which spans a body joint, wherein repeating (e.g. tessellated) elements in the mesh, grid, or lattice have quadrilateral (e.g. square, rectangular, rhomboid, diamond, parallelogram, or trapezoidal) shapes. In an example, an energy-conducting mesh can be made by laser cutting. In an example, an energy-conducting mesh, grid, or lattice which spans a body joint can be woven into an article of clothing. In an example, an energy-conducting mesh, grid, or lattice which spans a body joint can be printed onto an article of clothing.

In an example, an energy pathway can comprise an energy-conducting mesh, grid, or lattice which spans a body joint, wherein spaces or gaps in the mesh, grid, or lattice have hexagonal shapes (e.g. a honeycomb mesh). In an example, an energy pathway can comprise an energy-conducting mesh, grid, or lattice which spans a body joint, wherein repeating (e.g. tessellated) elements in the mesh, grid, or lattice have hexagonal shapes (e.g. a honeycomb mesh). In an example, a plurality of energy pathways which span a body joint can be woven together into an energy-conducting mesh, grid, or lattice.

In an example, a plurality of energy pathways which span a body joint can collectively comprise a hexagonal (e.g. honeycomb) energy-conducting mesh, grid, or lattice. In an example, an energy pathway can comprise an energy-conducting mesh, grid, or lattice with a tessellating pattern. In an example, an energy pathway can comprise an elastic energy-conducting mesh, grid, or lattice which spans a body joint.

In an example, an energy pathway can comprise an energy-conducting mesh, grid, or lattice which spans a body joint, wherein spaces or gaps in the mesh, grid, or lattice have quadrilateral (e.g. square, rectangular, rhomboid, diamond, parallelogram, or trapezoidal) shapes. In an example, a plurality of energy pathways which span a body joint can collectively comprise an energy-conducting mesh, grid, or lattice. In an example, an energy pathway can comprise an energy-conducting mesh, grid, or lattice which spans a body joint.

In an example, energy emitters at different locations along an energy pathway can be activated at different times to measure energy transmission in different directions. In an example, a plurality of energy pathways can span the same body joint, wherein energy transmissions through these pathways are multiplexed, wherein energy emitters in different pathways are activated at different times to measure energy transmissions in different directions and/or across different distances.

In an example, energy pulses can be transmitted through one or more energy pathways. In an example, transmission of energy through an energy pathway spanning a joint can be triggered (e.g. activated) in response to analysis of data from an inertial-based motion sensor. In an example, an energy pathway spanning a joint can be triggered (e.g. activated) based on data from an inertial-based motion sensor. In an example, energy emitters at different locations along an energy pathway can be activated at different times to measure energy transmission across different distances. In an example, a plurality of energy pathways can span a body joint, wherein energy is constantly transmitted through a first energy pathway, wherein energy is not constantly transmitted through the second energy pathway, and wherein energy transmission is triggered (e.g. activated) through the second energy pathway in response to analysis of data from the first energy pathway.

In an example, a plurality of energy pathways can span the same body joint, wherein energy is constantly transmitted through a first energy pathway, but energy is not constantly transmitted through a second energy pathway. In an example, energy emitters can emit energy pulses through an energy pathway. In an example, a plurality of energy pathways span a body joint, wherein transmission of energy through a second energy pathway is triggered (e.g. activated) in response to analysis of data from a first energy pathway. In an example, an array of energy emitters can be sequentially activated to transmit energy through different lengths of an energy pathway.

In an example, a plurality of energy pathways can span the same body joint, wherein energy emitters in different pathways are activated at different times to measure energy transmission in different directions and/or across different distances, and wherein joint analysis of energy transmissions in these different directions and/or across these different distances provides more accurate measurement of joint configuration and motion. In an example, a plurality of energy pathways span a body joint, wherein transmission of energy through a (more energy-intensive) second energy pathway is triggered (e.g. activated) in response to analysis of data from a (less energy-intensive) first energy pathway.

In an example, there can be a plurality of energy emitters in energy communication with an energy pathway which spans a body joint, wherein a first energy emitter emits energy with a first intensity level and a second energy emitter emits energy with a second intensity level. In an example, there can be a plurality of energy emitters in energy communication with an energy pathway which spans a body joint, wherein a first energy emitter emits energy at a first time and a second energy emitter emits energy at a second time. In an example, an energy emitter can transmit energy into a first end of a looping and/or U-shaped energy pathway which is distal relative to a body joint and an energy receiver can receive energy transmitted through the pathway from a second end of the pathway which is also distal relative to the body joint.

In an example, there can be a plurality of energy emitters in energy communication with an energy pathway which spans a body joint, wherein a first energy emitter emits energy for a first duration (e.g. time interval) and a second energy emitter emits energy for a second duration (e.g. time interval). In an example, there can be two energy emitters at two different locations on an energy pathway on the same side (e.g. proximal side or distal side) of a joint. In an example, there can be two energy emitters at two different locations on an energy pathway on different sides (e.g. proximal side and distal side) of a joint.

In an example, multiple energy emitters can transmit energy into different locations on an energy pathway. In an example, there can be two energy receivers at two different locations on an energy pathway on the same side (e.g. proximal side or distal side) of a joint. In an example, there can be two energy receivers at two different locations on an energy pathway on different sides (e.g. proximal side and distal side) of a joint. In an example, multiple energy receivers at different locations along the length of an energy pathway can be used to measure energy transmission over different distances.

In an example, multiple energy receivers can receiver energy from different locations on an energy pathway. In an example, an energy emitter can transmit energy into a first end of a looping and/or U-shaped energy pathway and an energy receiver can receive energy transmitted through the pathway from a second end of this pathway. In an example, an energy emitter can transmit energy into multiple energy pathways. In an example, there can be a plurality of energy emitters in energy communication with an energy pathway which spans a body joint, wherein a first energy emitter emits energy with a first wavelength and/or frequency and a second energy emitter emits energy with a second wavelength and/or frequency.

In an example, an energy emitter can transmit energy into one end of an energy pathway and an energy receiver can receive energy transmitted through the pathway from the opposite end of the pathway. In an example, multiple energy emitters at different locations along the length of an energy pathway can be used to measure energy transmission over different distances. In an example, an energy emitter can transmit energy into a first end of a looping and/or U-shaped energy pathway which is proximal relative to a body joint and an energy receiver can receive energy transmitted through the pathway from a second end of the pathway which is also proximal relative to the body joint. In an example, an energy receiver can receive energy from multiple energy pathways.

In an example, one or more energy pathways can be attached and detached to different locations on an article of clothing, enabling customization of the pathways and/or motion recognition clothing for a specific person or type of activity. In an example, one or more energy receivers can be moved to different areas on an energy pathway to customize the pathway configuration for a specific person or type of activity. In an example, an article of clothing can have a channel, lumen, and/or pocket into which an energy pathway can be removably inserted (and removed for washing the clothing).

In an example, the locations of one or more energy pathways can be moved on different areas of an article of clothing, enabling customization of the pathways and/or motion recognition clothing for a specific person or type of activity. In an example, energy pathways, energy emitters, and/or energy receivers can be snapped or otherwise removably-connected to different locations an article of clothing. In an example, one or more energy emitters can be attached and detached to different locations on an energy pathway to customize the pathway configuration for a specific person or type of activity.

In an example, one or more energy emitters can be moved to different areas on an energy pathway to customize the pathway configuration for a specific person or type of activity. In an example, a first configuration of modular energy pathways can be optimal for capturing body motion during running, a second configuration of modular energy pathways can be optimal for capturing body motion during swimming, and a third configuration of modular energy pathways can be optimal for measuring the random twitches of a couch potato. In an example, an energy pathway, energy emitter, and/or energy receiver can be temporarily removed from an article of clothing so that the clothing can be washed without possibly harming them.

In an example, one or more energy receivers can be attached and detached to different locations on an energy pathway to customize the pathway configuration for a specific person or type of activity. In an example, energy emitters and/or energy receivers can be snapped or otherwise removably-connected to different locations on an energy pathway. In an example, energy pathways, energy emitters, and/or energy receivers can be modular and/or moveable. In an example, a plurality of modular energy pathways can be inserted into and/or longitudinally shifted within different channels, lumens, or pockets in an article of clothing in order to measure body configuration and motion using different pathway configurations. In an example, energy emitters and/or energy receivers can be snapped or otherwise removably-connected to different locations an energy-conducting mesh, grid, or lattice.

In an example, the type of energy which is transmitted through an energy pathway can be electrical energy, light energy, or sound energy. In an example, an energy pathway can be an electrical energy pathway. In an example, an energy pathway can be a light energy pathway. In an example, an energy pathway can be a sonic energy pathway.

In an example, a wearable device for measuring (e.g. modeling) body changes in body configuration and/or motion can comprise: at least one flexible electrical energy pathway (e.g. electroconductive channel) which spans at least one body joint; at least one electrical energy emitter whose electrical energy is directed into the at least one pathway; and at least one electrical energy receiver which receives electrical energy from the at least one electrical energy emitter after the electrical energy has been transmitted through the at least one pathway; and a data processor, wherein changes in the configuration (e.g. configuration or configurations) of the at least one body joint cause changes in the shape (e.g. shape or shapes) of the at least one pathway, wherein changes in the shape (e.g. shape or shapes) of the at least one pathway cause changes in attributes (e.g. parameters) of the electrical energy transmitted through the at least one pathway, and wherein changes in the attributes (e.g. parameters) of the electrical energy are analyzed in the data processor to measure (e.g. model) changes in the configuration (e.g. configuration or configurations) of the at least one body joint.

In an example, changes in the resistance of an electrical energy pathway spanning a body joint which are caused by deformation (e.g. elongation or bending) of the pathway can be used to measure joint configuration and motion. In an example, changes in the capacitance of an electrical energy pathway spanning a body joint which are caused by deformation (e.g. elongation or bending) of the pathway can be used to measure joint configuration and motion. In an example, changes in the electrical current transmitted through an electrical energy pathway spanning a body joint which are caused by deformation (e.g. elongation or bending) of the pathway can be used to measure joint configuration and motion. In an example, changes in the impedance of an electrical energy pathway spanning a body joint which are caused by deformation (e.g. elongation or bending) of the pathway can be used to measure joint configuration and motion. In an example, one or more parameters of electrical energy transmitted through an electrical energy pathway can be selected from the group consisting of: resistance, capacitance, current, and impedance.

In an example, an electrical energy pathway can be made with nickel, silver, gold, copper, or aluminum. In an example, an electrical energy pathway can comprise at least three layers, wherein first and third layers are electroconductive and a second layer between them is not electroconductive. In an example, an electrical energy pathway can comprise electroconductive fibers, yarns, threads, strands, substrates, layers, or textiles. In an example, an electrical energy pathway can comprise undulating (e.g. sinusoidal or zigzag) electroconductive yarn. In an example, a capacitive electrical energy pathway can comprise at least three layers, wherein first and third layers are electroconductive and a second layer between them is not electroconductive.

In an example, an electrical energy pathway can comprise a plurality of electroconductive layers. In an example, an electrical energy pathway can comprise a plurality of layers, wherein a subset of these layers are electroconductive and a subset of these layer are not electroconductive. In an example, an electrical energy pathway can comprise a stretchable dielectric material between first and second conductive layers. In an example, an electrical energy pathway can comprise a elastomeric polymer which has been doped, impregnated, and/or coated with conductive material. In an example, an electrical energy pathway can comprise a plurality of parallel electroconductive layers.

In an example, a wearable device for measuring (e.g. modeling) body changes in body configuration and/or motion can comprise: at least one flexible light energy pathway (e.g. waveguide) which spans at least one body joint; at least one light energy emitter whose light is directed into the at least one pathway; and at least one light energy receiver which receives light from the at least one light energy emitter after the light has been transmitted through the at least one pathway; and a data processor, wherein changes in the configuration (e.g. configuration or configurations) of the at least one body joint cause changes in the shape (e.g. shape or shapes) of the at least one pathway, wherein changes in the shape (e.g. shape or shapes) of the at least one pathway cause changes in attributes (e.g. parameters) of the light transmitted through the at least one pathway, and wherein changes in the attributes (e.g. parameters) of the light are analyzed in the data processor to measure (e.g. model) changes in the configuration (e.g. configuration or configurations) of the at least one body joint.

In an example, changes in the power or intensity of light transmitted through an light energy pathway spanning a body joint which are caused by deformation (e.g. elongation or bending) of the pathway can be used to measure joint configuration and motion. In an example, changes in the wavelength or spectral distribution of light transmitted through an light energy pathway spanning a body joint which are caused by deformation (e.g. elongation or bending) of the pathway can be used to measure joint configuration and motion. In an example, changes in the phase of light transmitted through an light energy pathway spanning a body joint which are caused by deformation (e.g. elongation or bending) of the pathway can be used to measure joint configuration and motion.

In an example, changes in the direction of light transmitted through an light energy pathway spanning a body joint which are caused by deformation (e.g. elongation or bending) of the pathway can be used to measure joint configuration and motion. In an example, one or more parameters of light energy transmitted through an light energy pathway can be selected from the group consisting of: light power or intensity, light wavelength or spectral distribution, light direction, and light phase.

In an example, a light energy emitter can be a light emitting diode (LED). In an example, a light energy emitter can be an organic light emitting diode (OLED). In an example, a light energy emitter can be an active matrix organic light-emitting diode (AMOLED). In an example, a light energy emitter can be a collimated light projector or laser. In an example, a light energy receiver can be a photodiode, photodetector, or photometer. In an example, a light energy receiver can be a variable-translucence sensor. In an example, a light energy receiver can be a spectroscopy sensor.

In an example, a light energy pathway can have a core with multiple light-conducting fibers. In an example, a light energy pathway can be made with nematic liquid crystalline material. In an example, a light energy pathway can comprise a plurality of Fiber Bragg Gratings with different periodicities. In an example, a light energy pathway can comprise a first substantially-transparent elastomeric material which is doped or impregnated with a second light-absorbing, light-reflecting, or light-polarizing material.

In an example, a light energy pathway can comprise a waveguide made from cladded elastomeric material with a high refractive index. In an example, a plurality of light energy pathways can span the same body member, wherein these pathways differ in grating periodicity. In an example, a light energy pathway can comprise a cladded light-conducting fiber. In an example, a light energy pathway can comprise a waveguide made from elastomeric material with a high refractive index. In an example, a light energy pathway can have variable longitudinal density.

In an example, a light energy pathway can be made with platinum-catalyzed silicone. In an example, a light energy pathway can comprise a plurality of Fiber Bragg Gratings at different locations on the pathway. In an example, a light energy pathway can have cross-sectional variation in refractive index. In an example, a light energy pathway can be doped or impregnated with light-reflecting particles or microstructures. In an example, a light energy pathway can comprise a longitudinal series of refractive elements which are separated by non-uniform distances.

In an example, a light energy pathway can comprise a waveguide with slots or slits through which light escapes. In an example, a plurality of light energy pathways can span the same body member, wherein these pathways differ in material. In an example, a light energy pathway can be made with polybutylene adipate terephthalate. In an example, a light energy pathway can comprise a plurality of refractive gratings with different orientations. In an example, a light energy pathway can have longitudinal variation in refractive index. In an example, a light energy pathway can be made with polybutylene adipate-co-terephthalate.

In an example, a light energy pathway can comprise a plurality of refractive gratings with different periodicities. In an example, a light energy pathway can have periodic variation in its refractive index. In an example, a light energy pathway can be made with polybutyrate. In an example, a light energy pathway can comprise a plurality of refractive gratings with different wavelengths. In an example, a light energy pathway can have periodic variation in its refractive index which interacts with light of a particular wavelength. In an example, a light energy pathway can be made a light-conducting elastomeric polymer which has been doped and/or impregnated with light-absorbing, light-reflecting, or light-polarizing material (e.g. dye or crystals).

In an example, a light energy pathway can comprise a non-linear Fiber Bragg Grating. In an example, a light energy pathway can comprise one or more refractive gratings. In an example, a light energy pathway can comprise a beam splitter. In an example, a light energy pathway can comprise a plurality of refractive gratings at different locations on the pathway. In an example, a light energy pathway can have radially-asymmetric cladding, wherein there is radially variation in cladding thickness.

In an example, a light energy pathway can comprise a bundle of eight light-conducting fibers. In an example, a light energy pathway can comprise a plurality of light-conducting fibers and slots, slits, or nicks which cause light to escape from one pathway to another pathway. In an example, a light energy pathway can have radially-asymmetric cladding, wherein there is radially variation in cladding smoothness. In an example, a light energy pathway can comprise a bundle of four light-conducting fibers. In an example, a light energy pathway can comprise a plurality of waveguides which are made from a silicone material such as polydimethylsiloxane (PDMS).

In an example, a light energy pathway can have radially-asymmetric cladding, wherein there is radially variation in cladding coverage. In an example, a light energy pathway can comprise a longitudinal series of slits, slots, or channels. In an example, a light energy pathway can comprise a waveguide which is made from a silicone material such as polydimethylsiloxane (PDMS) which has been dyed. In an example, a plurality of light energy pathways can span the same body member, wherein these pathways differ in elasticity. In an example, a light energy pathway can comprise a long period fiber grating. In an example, a light energy pathway can comprise a waveguide which is made from a silicone material such as polydimethylsiloxane (PDMS).

In an example, a plurality of light energy pathways can span the same body member, wherein these pathways differ in transparency. In an example, a light energy pathway can be made a transparent polymer which has been doped and/or impregnated with light-absorbing, light-reflecting, or light-polarizing material (e.g. dye or crystals). In an example, a light energy pathway can comprise a non-linear refractive grating. In an example, a light energy pathway can have (radially) asymmetric cladding.

In an example, a light energy pathway can be made from silicone material (e.g. polydimethylsiloxane) which has been doped and/or impregnated with light-absorbing, light-reflecting, or light-polarizing material (e.g. dye or crystals). In an example, a light energy pathway can comprise a plurality of Fiber Bragg Gratings with different orientations. In an example, a light energy pathway can have (radially) symmetric cladding. In an example a light energy pathway can have cross-sectional core eccentricity. In an example, a light energy pathway can comprise a longitudinal series of refractive elements.

In an example, a light energy pathway can comprise a waveguide with total internal reflection. In an example, a plurality of light energy pathways can span the same body member, wherein these pathways differ in length. In an example, a light energy pathway can be doped or impregnated with light-refracting particles or microstructures. In an example, a light energy pathway can comprise a longitudinal series of refractive elements which are separated by increasing distances. In an example, a light energy pathway can comprise an undulating hollow channel. In an example, deformation of a light energy pathway can change the direction of light transmitted through the pathway.

In an example, a light energy pathway can be made with photonic crystalline material. In an example, a light energy pathway can comprise a plurality of Fiber Bragg Gratings with different wavelengths. In an example, a light energy pathway can have an interferometery complex. In an example, a light energy pathway can be doped or impregnated with light-absorbing particles or microstructures. In an example, a light energy pathway can comprise a Mach-Zehnder interferometer. In an example, a light energy pathway can comprise multiple layers of polydimethylsiloxane, wherein a first subset of these layers are transparent and a second subset of these layers are dyed.

In an example, light energy transmitted through a light energy pathway can be split and then recombined. In an example, a light energy pathway can be made a light-conducting polymer which has been doped and/or impregnated with light-absorbing, light-reflecting, or light-polarizing material (e.g. dye or crystals). In an example, a light energy pathway can comprise a multi-core optical fiber. In an example, a light energy pathway can comprise one or more Fiber Bragg Gratings. In an example, there can be periodic and/or repeated longitudinal variation in the refractive index of a light energy pathway. In an example, a light energy pathway can comprise a Fabry-Perot interferometer.

In an example, a light energy pathway can comprise a waveguide made from cladded polydimethylsiloxane. In an example, a plurality of light energy pathways can span the same body member, wherein these pathways differ in refractive index. In an example a light energy pathway can have cross-sectional radial asymmetry. In an example, a light energy pathway can comprise a longitudinal series of equidistant refractive elements. In an example, a light energy pathway can comprise a waveguide with a heterogeneous refractive structure. In an example, a plurality of light energy pathways can span the same body member, wherein these pathways differ in cladding.

In an example, a light energy pathway can comprise a bundle of six light-conducting fibers. In an example, a light energy pathway can comprise a waveguide made from polydimethylsiloxane. In an example, a light energy pathway can have radially-asymmetric cladding, wherein there is radially variation in cladding shape. In an example, a light energy pathway can be doped or impregnated with light-polarizing particles or microstructures. In an example, a light energy pathway can comprise a Michelson interferometer.

In an example, a light energy pathway can comprise multiple light-conducting layers. In an example, there can be periodic and/or repeated longitudinal variation in the material of a light energy pathway. In an example, a light energy pathway can comprise a bundle of light-conducting fibers. In an example, a light energy pathway can comprise a Sagnac interferometer. In an example, a light energy pathway can have radially-asymmetric cladding, wherein there is radially variation in cladding refraction.

In an example, a wearable device for measuring (e.g. modeling) body changes in body configuration and/or motion can comprise: at least one flexible sonic energy pathway which spans at least one body joint; at least one sonic energy emitter whose sonic energy is directed into the at least one pathway; and at least one sonic energy receiver which receives sonic energy from the at least one sonic energy emitter after the sonic energy has been transmitted through the at least one pathway; and a data processor, wherein changes in the configuration (e.g. configuration or configurations) of the at least one body joint cause changes in the shape (e.g. shape or shapes) of the at least one pathway, wherein changes in the shape (e.g. shape or shapes) of the at least one pathway cause changes in attributes (e.g. parameters) of the sonic energy transmitted through the at least one pathway, and wherein changes in the attributes (e.g. parameters) of the sonic energy are analyzed in the data processor to measure (e.g. model) changes in the configuration (e.g. configuration or configurations) of the at least one body joint.

In an example, a sonic energy pathway can transmit ultrasonic energy. In an example, changes in the intensity or amplitude of sonic energy transmitted through a sonic energy pathway spanning a body joint which are caused by deformation (e.g. elongation or bending) of the pathway can be used to measure joint configuration and motion. In an example, changes in the frequency of sonic energy transmitted through a sonic energy pathway spanning a body joint which are caused by deformation (e.g. elongation or bending) of the pathway can be used to measure joint configuration and motion. In an example, changes in the waveform or phase of sonic energy transmitted through a sonic energy pathway spanning a body joint which are caused by deformation (e.g. elongation or bending) of the pathway can be used to measure joint configuration and motion.

In an example, a wearable device can comprise an energy source which provides power to an energy emitter, an energy receiver, and/or a data processor, wherein this energy source is a battery. In an example, a wearable device can comprise an energy source which provides power to an energy emitter, an energy receiver, and/or a data processor, wherein this energy source harvests, transduces, or generates electrical energy from ambient light energy. In an example, an energy pathway can include triboelectric sensors.

In an example, a wearable device can comprise an energy source which provides power to an energy emitter, an energy receiver, and/or a data processor, wherein this energy source harvests, transduces, or generates electrical energy from (body) thermal energy. In an example, a wearable device can be used by a tribe of eclectic censors. In an example, a wearable device can comprise an energy source which provides power to an energy emitter, an energy receiver, and/or a data processor, wherein this energy source harvests, transduces, or generates electrical energy from kinetic energy. In an example, an energy pathway can be piezoelectric and/or piezoresistive. In an example, a wearable device can comprise an energy source which provides power to an energy emitter, an energy receiver, and/or a data processor, wherein this energy source harvests, transduces, or generates electrical energy from ambient electromagnetic energy.

In an example, a wearable device to measure and/or recognize body configuration and/or motion can include a plurality of inertial motion sensors (e.g. Inertial Measurement Units or IMUs). In an example an inertial motion sensor can comprise one or more sensors selected from the group consisting of: an accelerometer, a gyroscope, and an inclinometer In an example, a wearable device to measure and/or recognize body configuration and/or motion can include a plurality of energy pathways which span a body joint and a plurality of inertial motion sensors (e.g. Inertial Measurement Units or IMUs) on either side of the body joint.

In an example, a wearable device to measure and/or recognize body configuration and/or motion can include a plurality of energy pathways which span a body joint and a plurality of inertial motion sensors (e.g. Inertial Measurement Units or IMUs) on either side of the body joint, wherein data from the energy pathways and data from the inertial motion sensors is jointly analyzed to measure and/or recognize joint configuration and/or motion. In an example, a wearable device to measure and/or recognize body configuration and/or motion can include a plurality of energy pathways which span the same body joint and a plurality of inertial motion sensors (e.g. Inertial Measurement Units or IMUs) located proximally and distally relative to that body joint, wherein data from the energy pathways and data from the inertial motion sensors is jointly analyzed to measure and/or recognize joint configuration and/or motion.

In an example, a wearable device for measuring body configuration and/or motion can further comprise an electroencephalography (EEG) sensor. In an example, a wearable device for measuring body configuration and/or motion can further comprise a heart rate monitor. In an example, a wearable device for measuring body configuration and/or motion can further comprise a respiration or pulmonary function monitor. In an example, a wearable device for measuring body configuration and/or motion can further comprise an oximetry sensor. In an example, a wearable device for measuring body configuration and/or motion can further comprise an electromyography (EMG) sensor.

In an example, a wearable device for measuring body configuration and/or motion can further comprise an inclinometer or tilt sensor. In an example, a wearable device for measuring body configuration and/or motion can further comprise an ambient light sensor. In an example, a wearable device for measuring body configuration and/or motion can further comprise a vibration sensor. In an example, a wearable device for measuring body configuration and/or motion can further comprise a force sensors.

In an example, a wearable device for measuring body configuration and/or motion can further comprise an infrared light sensor. In an example, a wearable device for measuring body configuration and/or motion can further comprise a galvanic skin response (GSR) sensor. In an example, a wearable device for measuring body configuration and/or motion can further comprise a magnometer. In an example, a wearable device for measuring body configuration and/or motion can further comprise a glucose sensor.

In an example, a wearable device for measuring body configuration and/or motion can further comprise a microphone. In an example, a wearable device for measuring body configuration and/or motion can further comprise a chemical sensor. In an example, a wearable device for measuring body configuration and/or motion can further comprise a GPS or other location sensor. In an example, a wearable device for measuring body configuration and/or motion can further comprise a gyroscope. In an example, a wearable device for measuring body configuration and/or motion can further comprise a Hall-effect sensor.

In an example, a wearable device for measuring body configuration and/or motion can further comprise a temperature sensor. In an example, a wearable device for measuring body configuration and/or motion can further comprise a spectroscopy sensor. In an example, a wearable device for measuring body configuration and/or motion can further comprise a chemoreceptor.

In an example, a wearable device for measuring body configuration and/or motion can further comprise an electrocardiogram (ECG) sensor. In an example, a wearable device for measuring body configuration and/or motion can further comprise an accelerometer. In an example, a wearable device for measuring body configuration and/or motion can further comprise a biochemical sensor. In an example, a wearable device for measuring body configuration and/or motion can further comprise an electromagnetic field sensor.

In an example, a wearable device for measuring body configuration and/or motion can further comprise a humidity sensor. In an example, a wearable device for measuring body configuration and/or motion can further comprise a blood pressure sensor. In an example, a wearable device for measuring body configuration and/or motion can further comprise a capacitive sensor. In an example, a wearable device for measuring body configuration and/or motion can further comprise a pressure sensor.

In an example, a wearable device for measuring body configuration and/or motion can further comprise an acoustic sensor. In an example, a wearable device for measuring body configuration and/or motion can further comprise a moisture sensor. In an example, a wearable device for measuring body configuration and/or motion can further comprise a camera. In an example, a wearable device for measuring body configuration and/or motion can further comprise a microphone.

In an example, a wearable device can further comprise a data processor. In an example, a wearable device can further comprise a wireless data receiver. In an example, a wearable device can further comprise a wireless data transmitter. In an example, a wearable device can further comprise a display screen. In an example, a wearable device can further comprise an electromagnetic actuator. In an example, a wearable device can further comprise a global positioning system (“GPS”) component. In an example, a wearable device can further comprise an image projector.

In an example, a wearable device can further comprise an infrared light emitter. In an example, a wearable device can further comprise a keypad or keyboard. In an example, a wearable device can further comprise a LED array. In an example, a wearable device can further comprise a microphone. In an example, a wearable device can further comprise a neurostimulator. In an example, a wearable device can further comprise a speaker. In an example, a wearable device can further comprise a spectroscopic sensor. In an example, a wearable device can further comprise a touch screen. In an example, a wearable device can further comprise a vibrating or other tactile sensation creating component.

In an example, a wearable device with a plurality of energy pathways spanning the same body joint can create redundant data, which enables accurate measurement of joint configuration and motion even if some of the energy pathways generate erroneous results due to shifting of the device. In an example, a wearable device with a plurality of energy pathways spanning the same body joint at different angles can provide more accurate measurement of joint configuration and motion than a device with only one energy pathway or multiple pathways spanning the joint at the same angle.

In an example, a first energy pathway spanning a body joint can provide more accurate measurement of joint motion over a first range of motion and a second energy pathway can provide more accurate measurement of joint motion over a second range of motion, so multivariate (e.g. joint, combined, or integrated) analysis of data from both pathways provides more accurate measurement of joint motion than either pathway alone. In an example, multivariate (e.g. joint, combined, or integrated) analysis of data from a plurality of energy pathways spanning the same body joint can provide more accurate measurement of joint configuration and motion.

In an example, multivariate (e.g. joint, combined, or integrated) analysis of data from a plurality of energy pathways spanning the same body joint can provide more accurate measurement of joint configuration and motion in case some of the pathways generate erroneous results. In an example, multivariate (e.g. joint, combined, or integrated) analysis of data from a plurality of energy pathways spanning the same body joint can provide more accurate measurement of joint configuration and motion in case some of the pathways generate erroneous results due to shifting of a wearable device and/or gaps between the device and a body member containing the joint.

In an example, a wearable device with a plurality of energy pathways spanning the same body joint can provide more accurate measurement of joint configuration and motion. In an example, a wearable device with a plurality of energy pathways spanning the same body joint can provide more accurate measurement of joint configuration and motion in case some of the pathways generate erroneous results. In an example, a wearable device with a plurality of energy pathways spanning the same body joint can provide more accurate measurement of joint configuration and motion in case some of the pathways generate erroneous results due to shifting of the device and/or gaps between the device and a body member containing the joint.

In an example, a device can comprise at least two energy pathways which span the same body joint, wherein there is continuous energy flow through a first energy pathway and wherein energy flow through a second energy pathway only occurs when triggered based on analysis of data from the first energy pathway. In an example, a first energy pathway spanning a body joint can provide more accurate measurement of fast joint motion and a second energy pathway can provide more accurate measurement of slow joint motion, so multivariate (e.g. joint, combined, or integrated) analysis of data from both pathways provides more accurate measurement of joint motion than either pathway alone.

In an example, a wearable device with a plurality of energy pathways spanning the same body joint in different directions can provide more accurate measurement of joint configuration and motion than a device with only one energy pathway spanning the joint or with multiple pathways spanning the joint in the same direction. In an example, a first energy pathway spanning a body joint can provide more accurate measurement of a single joint movement and a second energy pathway can provide more accurate measurement of repeated joint movement, so multivariate (e.g. joint, combined, or integrated) analysis of data from both pathways provides more accurate measurement of joint motion than either pathway alone. In an example, a first energy pathway spanning a body joint can provide more accurate measurement of joint bending and a second energy pathway can provide more accurate measurement of joint torsion, so multivariate (e.g. joint, combined, or integrated) analysis of data from both pathways provides more accurate measurement of joint motion than either pathway alone.

In an example, a device can comprise first and second energy pathways which span the same body joint, wherein a first type of energy is transmitted through the first pathway and a second type of energy is transmitted through the second pathway, wherein there is continuous energy flow through the first energy pathway, and wherein energy flow through the second energy pathway only occurs when triggered based on analysis of data from the first energy pathway. In an example, a first energy pathway spanning a body joint can provide more accurate measurement of joint movement in a first direction and a second energy pathway can provide more accurate measurement of joint movement in a second direction, so multivariate (e.g. joint, combined, or integrated) analysis of data from both pathways provides more accurate measurement of joint motion than either pathway alone.

In an example, data from one or more energy receivers can be analyzed using eigenvalue decomposition to measure body configuration or motion. In an example, data from one or more energy receivers can be analyzed using factor analysis to measure body configuration or motion. In an example, data from multiple pathways can be analyzed to identify and compensate for a device shifting or sliding on a person's body. In an example, data from one or more energy receivers can be analyzed using discriminant analysis to measure body configuration or motion.

In an example, data from one or more energy receivers can be analyzed using Markov modelling to measure body configuration or motion. In an example, data from one or more energy receivers can be analyzed using multivariate analysis to measure body configuration or motion. In an example, data from one or more energy receivers can be analyzed using Bayesian statistical methods to measure body configuration or motion. In an example, data from one or more energy receivers can be analyzed using least squares estimation to measure body configuration or motion.

In an example, data from one or more energy receivers can be analyzed using principal components analysis to measure body configuration or motion. In an example, data from one or more energy receivers can be analyzed using random forest analysis to measure body configuration or motion. In an example, data from one or more energy receivers can be analyzed using kinematic modeling to measure body configuration or motion. In an example, data from one or more energy receivers can be analyzed using decision tree analysis to measure body configuration or motion. In an example, data from multiple pathways can be analyzed to identify and compensate for measurement errors in a subset of those pathways.

In an example, data from multiple pathways can be analyzed to identify and compensate for a device shifting or sliding around the circumference of a body member. In an example, data from one or more energy receivers can be analyzed using Fourier analysis to measure body configuration or motion. In an example, data from one or more energy receivers can be analyzed using artificial intelligence and/or machine learning to measure body configuration or motion. In an example, data from one or more energy receivers can be analyzed using auto-regression to measure body configuration or motion. In an example, data from multiple pathways can be analyzed to identify and compensate for a device shifting or sliding relative to a body joint. In an example, data from one or more energy receivers can be analyzed using an artificial neural network to measure body configuration or motion.

In an example, a wearable device for measuring body configuration and motion can be recalibrated after a selected number of joint extension and contraction cycles. In an example, a wearable device for measuring body configuration and motion can be (re)calibrated when it is first worn by a specific person in order to be customized to that person's specific anatomy and/or body kinetics. In an example, a wearable device for measuring body configuration and motion can be recalibrated at selected usage time intervals. In an example, a wearable device for measuring body configuration and motion can be recalibrated based on a change in environmental factors (such as temperature, humidity, GPS location, or atmospheric pressure).

In an example, a wearable device can comprise a plurality of energy pathways which span a joint and a plurality of inertial motion sensors, wherein data from the energy pathways is used to recalibrate results from the inertial motions sensors (e.g. to correct for drift). In an example, a wearable device for measuring body configuration and motion can be recalibrated each time a particular sequence of movements occurs. In an example, a wearable device for measuring body configuration and motion can be recalibrated to control for shifts in how energy pathways span a body joint.

In an example, a wearable device for measuring body configuration and motion can be recalibrated to control for changes in environmental conditions such as temperature. In an example, a wearable device can comprise a plurality of energy pathways which span a joint and a plurality of inertial motion sensors, wherein data from the inertial motion sensors is used to recalibrate results from the energy pathways. In an example, a wearable device for measuring body configuration and motion can be recalibrated to control for changes in how material responses to bending, stretching, or elongation with repeated motions.

In this disclosure, the term “proximal” refers to locations in (or on) a person's body which are closer to the person's heart via their cardiovascular system and the term “distal” refers to locations in (or on) a person's body which are farther from the person's heart via their cardiovascular system. This concludes the introductory section of this disclosure, which now moves on to discussing the embodiment of this invention shown in FIG. 1.

FIG. 1 shows an example of how this invention can be embodied in a wearable two-piece set of Motion Recognition Clothing™ which measures major joint motion and/or configuration for virtually the entire body via multiple sets of energy pathways. Each set of energy pathways spans a major body joint. Collecting and analyzing data from multiple redundant energy pathways spanning the same body joint provides more accurate measurement of the motion and/or configuration of that joint. In an example, each energy pathway is in energy communication with an energy emitter (e.g. energy input component) which directs energy into the energy pathway and with an energy receiver (e.g. energy sensor) which measures energy flow through the energy pathway.

In an example, energy pathways can transmit electromagnetic energy and changes in the transmission of electromagnetic energy can be used to measure joint motion and/or configuration. In an example, energy pathways can transmit light energy and changes in the transmission of light energy can be used to measure joint motion and/or configuration. In an example, energy pathways can transmit sonic energy and changes in sonic energy transmission can be used to measure joint motion and/or configuration. In an example, energy can be transmitted across a body joint in a generally distal-to-proximal direction. In another example, energy can be transmitted across a body joint in a generally proximal-to-distal direction. Changes in the configurations of the major body joints change the configurations of the energy pathways which, in turn, change the energy transmissions measured by the energy receivers (e.g. energy sensors) which, in turn, are used to estimate the motions and/or configurations of the body joints. In this example, energy pathways spanning multiple body joints collectively enable minimally-intrusive, ambulatory full-body motion capture.

FIG. 1 shows an example of an upper-body garment for measuring body joint motion and configuration comprising: an upper-body garment worn by a person, wherein the garment further comprises; a set of energy pathways which span the person's right elbow; a set of proximally-diverging energy pathways which span the person's right shoulder; a set of energy pathways which span the person's left elbow; a set of proximally-diverging energy pathways which span the person's left shoulder; a set of energy pathways which span a portion of the person's torso or back; a set of energy emitters (e.g. energy input components); and a set of energy receivers (e.g. energy sensors); wherein each energy pathway is in energy communication with an energy emitter (e.g. energy input component) which directs energy into the energy pathway and with an energy receiver (e.g. energy sensor) which measures energy transmission through the energy pathway; and wherein changes in the transmission of energy through energy pathways are analyzed to measure the motion and configuration of body joints.

FIG. 1 also shows an example of a lower-body garment for measuring body joint motion and configuration comprising: a lower-body garment worn by a person, wherein the garment further comprises; a set of energy pathways which span the person's right knee; a set of proximally-diverging energy pathways which span the person's right hip; a set of energy pathways which span the person's left knee; a set of proximally-diverging energy pathways which span the person's left hip; a set of energy emitters (e.g. energy input components); and a set of energy receivers (e.g. energy sensors); wherein each energy pathway is in energy communication with an energy emitter (e.g. energy input component) which directs energy into the energy pathway and with an energy receiver (e.g. energy sensor) which measures energy transmission through the energy pathway; and wherein changes in the transmission of energy through energy pathways are analyzed to measure the motion and configuration of body joints.

With respect to specific components, the example shown in FIG. 1 comprises a two-piece set of motion recognition clothing with an upper-body component (e.g. a shirt or top) 1006 and a lower-body component (e.g. a pair of pants) 1010. In an example, the upper-body component can be a sweat shirt with sets of embedded energy pathways. In an example, the upper-body component can be the upper piece of a sports uniform with sets of embedded energy pathways. The upper-body (shirt) component of this motion recognition clothing measures the motion and/or configuration of the wearer's elbows, shoulders, and torso and/or back. In an example, the lower-body component can be a pair of sweat pants with sets of embedded energy pathways. In an example, the lower-body component can be the lower piece of a sports uniform with sets of embedded energy pathways. The lower-body (pants) component of this motion recognition clothing measures the motion and/or configuration of the wearer's hips and knees.

With respect to specific components, the example shown in FIG. 1, the upper-body (shirt) component also comprises: a set of substantially-parallel energy pathways (including 1002) which span the person's right elbow in a longitudinal manner and are substantially evenly-spaced around the circumference of the person's elbow; a set of proximally-diverging energy pathways (including 1004) which span the person's right shoulder in a longitudinal manner and are substantially evenly-spaced around the circumference of the person's shoulder; a set of proximally-diverging energy pathways (including 1007) which span the person's left shoulder in a longitudinal manner and are substantially evenly-spaced around the circumference of the person's shoulder; a set of substantially-parallel energy pathways (including 1008) which span the person's left elbow in a longitudinal manner and are substantially evenly-spaced around the circumference of the person's elbow; and a set of energy pathways (including 1009) which span a portion of the person's torso and/or back. In an example, this clothing could be extended to also span the person's wrists, fingers, neck, and/or head.

In the example shown in FIG. 1, the lower-body (pants) component also includes: a set of proximally-diverging energy pathways (including 1013) which span the person's right hip in a longitudinal manner and are substantially evenly-spaced around a portion of the circumference of the person's hip; a set of substantially-parallel energy pathways (including 1011) which span the person's right knee in a longitudinal manner and are substantially evenly-spaced around the circumference of the person's knee; a set of proximally-diverging energy pathways (not visible in this FIGURE) which span the person's left hip in a longitudinal manner and are substantially evenly-spaced around a portion of the circumference of the person's hip; and a set of substantially-parallel energy pathways (including 1012) which span the person's left knee in a longitudinal manner and are substantially evenly-spaced around the circumference of the person's knee. In an example, this clothing could be extended to also span the person's ankles and/or feet.

The example shown in FIG. 1 also comprises a wrist-worn component 1001 and a torso-worn component 1014. In an alternative example, the latter component could be a hip-worn component that is worn on the person's hip as part of the lower-body component of the motion recognition clothing. As symbolically represented by lightning bolt symbols, the wrist-worn component 1001 and/or torso-worn component 1014 can be in wired and/or wireless communication with energy receivers (e.g. energy sensors) (including 1005), with energy emitters (e.g. energy input components) (including 1003), and/or with each other. The exact configuration of wires or other electrically-conductive connections to sensors or energy emitters (e.g. energy input components) is not central to this invention and wires are not shown in this FIGURE.

In an example, clothing with embedded energy pathways and a wrist-worn device can together comprise a system for measuring full-body motion and/or configuration. In an example, clothing with embedded energy pathways and a wrist-worn device can together comprise a system for minimally-intrusive, ambulatory full-body motion capture. In an example, clothing with embedded energy pathways and a wrist-worn device which are in wireless communication with each other can together comprise a system of motion recognition clothing.

In an alternative example, clothing with embedded energy pathways and electronically-functional eyewear can together comprise a system for measuring full-body motion and/or configuration. In an example, clothing with embedded energy pathways and electronically-functional eyewear can together comprise a system for minimally-intrusive, ambulatory full-body motion capture. In an example, clothing with embedded energy pathways and electronically-functional eyewear which are in wireless communication with each other can together comprise a system of motion recognition clothing.

In an example, a wrist-worn component and/or a torso-worn component can further comprise one or more sub-components selected from the group consisting of: a data processing component, a data communication component, a power source, a human-to-computer user interface, a computer-to-human interface, and a digital memory. In an example, a data control unit can be temporarily detached so that the remaining wearable portion of the invention can be washed.

In an example, a data processing component of this device can perform one or more functions selected from the group consisting of: amplify sensor signals, analyze data, analyze sensor information, convert analog signals to digital signals, determine a functional relationship between signal variation and joint angle variation, estimate joint angle, filter signals, model joint configuration, record data, run software applications, run software programs, and store data in memory.

In an example, a data communication component of this device can perform one or more functions selected from the group consisting of: transmit and receive data via Bluetooth, WiFi, Zigbee, or other wireless communication modality; transmit and receive data to and from a mobile electronic device such as a cellular phone, mobile phone, smart phone, electronic tablet; transmit and receive data to and from a separate wearable device such as a smart watch or electronically-functional eyewear; transmit and receive data to and from the internet; send and receive phone calls and electronic messages; transmit and receive data to and from a home appliance and/or home control system; and transmit and receive data to and from an implantable medical device.

In an example, a method for measuring, modeling, and/or capturing a person's shoulder motion and/or configuration can comprise: (a) measuring a first energy transmission from a first wearable energy pathway that is configured to span the portion of a person's body which contains their shoulder; (b) measuring a second energy transmission from a second wearable energy pathway that is configured to span the portion of a person's body which contains their shoulder; and (c) jointly analyzing the first and second energy transmissions in order to estimate, measure, and/or model the abduction, adduction, extension, flexion, and/or rotation of their shoulder.

In an example, first and second energy transmissions can be electrical energy. In an example, electrical energy can be conducted through the energy pathways and the amounts of electrical energy conducted can change when the configurations of the pathways change as the shoulder moves. In an example, electrical voltage, current, resistance, and/or impedance can be measured. In an example, first and second energy transmissions can be light energy. In an example, the energy pathways can be fiber optic. In an example, the amount, wavelength, and/or spectrum of light energy transmitted through the energy pathways can change when the configurations of the pathways change as the shoulder moves. In an example, the first and second energy transmissions can be sound energy. In an example, the energy transmissions can be ultrasonic. In an example, the amount, frequency, or pattern of sound energy transmitted through the energy pathways can change when the shapes of the pathways change.

In an example, joint statistical analysis of the first and second energy transmissions can provide more accurate estimation, measurement, and/or modeling of abduction, adduction, extension, flexion, and/or rotation of the person's shoulder than does separate statistical analysis of the first energy transmission or the second energy transmission. In an example, energy transmissions from the first and second energy pathways can be averaged together to reduce the variability of measurement and/or reduce the impact of measurement error in one pathway. In an example, a statistical method can be used which gives greater statistical weight to the first energy transmission over a first range of abduction, adduction, extension, flexion, and/or rotation and gives greater statistical weight to the second energy transmission over a second range of abduction, adduction, extension, flexion, and/or rotation. In an example, a statistical method can analyze differences between the first and second energy transmissions to determine if the locations of the wearable energy pathways relative to the surface of the person's body have shifted and to adjust estimation if such shifting occurs.

In an example, the relationship between energy transmission and shoulder configuration can be nonlinear and/or stochastic. In an example, joint analysis of the first and second energy transmissions from the first and second energy pathways spanning a person's shoulder can be done using one or more statistical methods selected from the group consisting of: multivariate linear regression or least squares estimation; factor analysis; Fourier Transformation; mean; median; multivariate logit; principal components analysis; spline function; auto-regression; centroid analysis; correlation; covariance; decision tree analysis; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian statistical method; chi-squared; eigenvalue decomposition; logit model; machine learning; power spectral density; power spectrum analysis; and/or probit model.

In an example, this invention can comprise first and second energy pathways which have longitudinal axes which span a person's shoulder. In an example, these longitudinal axes can be separated by a substantially constant percentage of the cross-sectional circumference of the person's shoulder. In an example, the first and second energy pathways are substantially parallel as they longitudinally span a distal skeletal member of a shoulder joint and diverge in a radial manner as they longitudinally span a proximal skeletal member of the shoulder joint. In an example, the first and second energy pathways can be substantially parallel as they longitudinally span the humerus and diverge as they longitudinally span the acromion, clavicle, coracoid process, and/or scapula; or vice versa. In an example, the first and second energy pathways can be pathways within an energy-transmitting mesh which spans the portion of a person's body which contains their shoulder joint.

In various examples, measurement of the configuration and movement of a person's shoulder can be especially useful for: athletic training and motion capture for sports which involve extensive arm motion (such as tennis and golf); rehabilitation for upper-body injuries and neurological impairment; measurement of caloric expenditure; ambulatory telerobotics; and upper-body avatar animation, computer gaming, and virtual reality.

In an example, the first and second energy pathways can be energy transmitting pathways which are incorporated into a shirt, other wearable top, shoulder tube, shoulder pad, or union suit. In an example, the first and second energy pathways can be woven into a shirt, other wearable top, shoulder tube, shoulder pad, or union suit. In an example, the first and second energy pathways can be sewn into, inserted into, or adhered to a shirt, other wearable top, shoulder tube, shoulder pad, or union suit. In an example, a shirt, other wearable top, shoulder tube, shoulder pad, or union suit can comprise part of a system of motion recognition clothing for measuring, modeling, and/or capturing changes in body motion and/or configuration. In an example, a data transmitting or processing component of such a system can be temporarily detached in order to wash the motion recognition clothing.

In an example, a method for measuring, modeling, and/or capturing a person's hip motion and/or configuration can comprise: (a) measuring a first energy transmission from a first wearable energy pathway that is configured to span the portion of a person's body which contains their hip; (b) measuring a second energy transmission from a second wearable energy pathway that is configured to span the portion of a person's body which contains their hip; and (c) jointly analyzing the first and second energy transmissions in order to estimate, measure, and/or model the abduction, adduction, extension, flexion, and/or rotation of their hip.

In an example, first and second energy transmissions can be electrical energy. In an example, this electrical energy can be conducted through the energy pathways and the amounts of electrical energy conducted can change when the configurations of the pathways change as the hip moves. In an example, electrical voltage, current, resistance, and/or impedance can be measured. In an example, the first and second energy transmissions can be light energy. In an example, the energy pathways can be fiber optic. In an example, the amount, wavelength, and/or spectrum of light energy transmitted through the energy pathways can change when the configurations of the pathways change as the hip moves. In an example, the first and second energy transmissions can be sound energy. In an example, the energy transmissions can be ultrasonic. In an example, the amount, frequency, or pattern of sound energy transmitted through the energy pathways can change when the shapes of the pathways change.

In an example, joint statistical analysis of the first and second energy transmissions can provide more accurate estimation, measurement, and/or modeling of abduction, adduction, extension, flexion, and/or rotation of the person's hip than does separate statistical analysis of the first energy transmission or the second energy transmission. In an example, energy transmissions from the first and second energy pathways can be averaged together to reduce the variability of measurement and/or reduce the impact of measurement error in one pathway. In an example, a statistical method can be used which gives greater statistical weight to the first energy transmission over a first range of abduction, adduction, extension, flexion, and/or rotation and gives greater statistical weight to the second energy transmission over a second range of abduction, adduction, extension, flexion, and/or rotation. In an example, a statistical method can analyze differences between the first and second energy transmissions to determine if the locations of the wearable energy pathways relative to the surface of the person's body have shifted and to adjust estimation if such shifting occurs.

In an example, the relationship between energy transmission and hip configuration can be nonlinear and/or stochastic. In an example, joint analysis of the first and second energy transmissions from the first and second energy pathways spanning a person's hip can be done using one or more statistical methods selected from the group consisting of: multivariate linear regression or least squares estimation; factor analysis; Fourier Transformation; mean; median; multivariate logit; principal components analysis; spline function; auto-regression; centroid analysis; correlation; covariance; decision tree analysis; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian statistical method; chi-squared; eigenvalue decomposition; logit model; machine learning; power spectral density; power spectrum analysis; and/or probit model.

In an example, this invention can comprise first and second energy pathways which have longitudinal axes which span a person's hip. In an example, the first and second energy pathways are substantially parallel as they longitudinally span a distal skeletal member of a hip joint and diverge in a radial manner as they longitudinally span a proximal skeletal member of the hip joint. In an example, the first and second energy pathways are substantially parallel as they longitudinally span the femur and diverge as they longitudinally span the Ilium; or vice versa. In an example, the first and second energy pathways can be concentric and/or nested as they span the portion of a person's body which contains a hip joint. In an example, the first and second energy pathways can be pathways within an energy-transmitting mesh which spans the portion of a person's body which contains their hip joint.

In various examples, measurement of the configuration and movement of a person's hip can be especially useful for: athletic training and motion capture for sports which involve extensive lower-body motion (such as bicycling and running); gait analysis, medical diagnosis, posture correction, and rehabilitation for injuries and neurological impairment; measurement of caloric expenditure (especially with respect to lower body motions that are not well measured by upper body motion sensors); ambulatory telerobotics; and lower-body avatar animation, computer gaming, and virtual reality.

In an example, the first and second energy pathways can be energy transmitting pathways which are incorporated into a pair of pants, shorts, hip pad, belt, or union suit. In an example, the first and second energy pathways can be woven into a pair of pants, shorts, hip pad, belt, or union suit. In an example, the first and second energy pathways can be sewn into, inserted into, or adhered to a pair of pants, shorts, hip pad, belt, or union suit. In an example, this pair of pants, shorts, hip pad, belt, or union suit can comprise part of a system of motion recognition clothing for measuring, modeling, and/or capturing changes in body motion and/or configuration. In an example, a data transmitting or processing component of such a system can be temporarily detached in order to wash the motion recognition clothing.

In an example, a method for measuring, modeling, and/or capturing a person's spine, back, and/or torso motion and/or configuration can comprise: (a) measuring a first energy transmission from a first wearable energy pathway that is configured to span the portion of a person's body which contains their back and/or torso; (b) measuring a second energy transmission from a second wearable energy pathway that is configured to span the portion of a person's body which contains their back and/or torso; and (c) jointly analyzing the first and second energy transmissions in order to estimate, measure, and/or model the abduction, extension, flexion, lateral bending, and/or rotation of their spine, back, and/or torso.

In an example, first and second energy transmissions can be electrical energy. In an example, this electrical energy can be conducted through the energy pathways and the amounts of electrical energy conducted can change when the configurations of the pathways change as the spine moves. In an example, electrical voltage, current, resistance, and/or impedance can be measured. In an example, the first and second energy transmissions can be light energy. In an example, the energy pathways can be fiber optic. In an example, the amount, wavelength, and/or spectrum of light energy transmitted through the energy pathways can change when the configurations of the pathways change as the spine moves. In an example, the first and second energy transmissions can be sound energy. In an example, the energy transmissions can be ultrasonic. In an example, the amount, frequency, or pattern of sound energy transmitted through the energy pathways can change when the shapes of the pathways change.

In an example, joint statistical analysis of the first and second energy transmissions can provide more accurate estimation, measurement, and/or modeling of abduction, extension, flexion, lateral bending, and/or rotation of the person's spine, back, and/or torso than does separate statistical analysis of the first energy transmission or the second energy transmission. In an example, energy transmissions from the first and second energy pathways can be averaged together to reduce the variability of measurement and/or reduce the impact of measurement error in one pathway. In an example, a statistical method can be used which gives greater statistical weight to the first energy transmission over a first range of abduction, extension, flexion, lateral bending, and/or rotation and gives greater statistical weight to the second energy transmission over a second range of abduction, extension, flexion, lateral bending, and/or rotation. In an example, a statistical method can analyze differences between the first and second energy transmissions to determine if the locations of the wearable energy pathways relative to the surface of the person's body have shifted and to adjust estimation if such shifting occurs.

In an example, the relationship between energy transmission and spine, back, and/or torso configuration can be nonlinear and/or stochastic. In an example, joint analysis of the first and second energy transmissions from the first and second energy pathways spanning a person's spine, back, and/or torso can be done using one or more statistical methods selected from the group consisting of: multivariate linear regression or least squares estimation; factor analysis; Fourier Transformation; mean; median; multivariate logit; principal components analysis; spline function; auto-regression; centroid analysis; correlation; covariance; decision tree analysis; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian statistical method; chi-squared; eigenvalue decomposition; logit model; machine learning; power spectral density; power spectrum analysis; and/or probit model.

In an example, this invention can comprise first and second energy pathways which have longitudinal axes which span a person's spine, back, and/or torso. In an example, these longitudinal axes can be separated by a substantially constant percentage of the cross-sectional circumference of the person's back. In an example, these longitudinal axes are substantially parallel when the back is straight. In an example, the first energy pathway can have a longitudinal axis which longitudinally spans the back and/or torso and the second energy pathway can span part of the cross-sectional perimeter of the back and/or torso. In an example, the first and second energy pathways are substantially parallel as they longitudinally spa lower spinal vertebrae and diverge in a radial manner as they longitudinally span higher spinal vertebrae. In an example, the first and second energy pathways are substantially parallel as they longitudinally span higher spinal vertebrae and diverge in a radial manner as they longitudinally spa lower spinal vertebrae. In an example, the first and second energy pathways can be concentric and/or nested as they span a person's back and/or torso. In an example, the first and second energy pathways can be pathways within an energy-transmitting mesh which spans a person's back and/or torso.

In various examples, measurement of the configuration and movement of a person's hip can be especially useful for: athletic training and motion capture for sports which involve extensive spinal motion; medical diagnosis, posture correction, and spinal injury avoidance; ambulatory telerobotics; and upper-body avatar animation, computer gaming, and virtual reality.

In an example, the first and second energy pathways can be energy transmitting pathways which are incorporated into a shirt, other top, torso and/or waist tube, torso and/or waist band, belt, bra, girdle, or union suit. In an example, the first and second energy pathways can be woven into a shirt, other top, torso and/or waist tube, torso and/or waist band, belt, bra, girdle, or union suit. In an example, the first and second energy pathways can be sewn into, inserted into, or adhered to a shirt, other top, torso and/or waist tube, torso and/or waist band, belt, bra, girdle, or union suit. In an example, this shirt, other top, torso and/or waist tube, torso and/or waist band, belt, bra, girdle, or union suit can comprise part of a system of motion recognition clothing for measuring, modeling, and/or capturing changes in body motion and/or configuration. In an example, a data transmitting or processing component of such a system can be temporarily detached in order to wash the motion recognition clothing. Example variations discussed elsewhere in this disclosure or in priority-linked disclosures can also be applied to this example where relevant.

In an example, an upper-body garment or article of clothing for measuring body configuration and motion can comprise: an upper-body garment or article of clothing worn by a person, wherein the garment or article of clothing further comprises; a set of energy pathways which span the person's right elbow; a set of proximally-diverging energy pathways which span the person's right shoulder; a set of energy pathways which span the person's left elbow; a set of proximally-diverging energy pathways which span the person's left shoulder; a set of energy pathways which span a portion of the person's torso or back; a plurality of energy emitters; and a plurality of energy receivers; wherein each energy pathway is in energy communication with an energy emitter which directs energy into the energy pathway and with an energy receiver which measures energy transmission through the energy pathway; and wherein changes in the transmission of energy through energy pathways are analyzed to measure the configuration and motion of body joints.

In an example, a lower-body garment or article of clothing for measuring body configuration and motion can comprise: a lower-body garment or article of clothing worn by a person, wherein the garment or article of clothing further comprises; a set of energy pathways which span the person's right knee; a set of proximally-diverging energy pathways which span the person's right hip; a set of energy pathways which span the person's left knee; a set of proximally-diverging energy pathways which span the person's left hip; a plurality of energy emitters; and a plurality of energy receivers; wherein each energy pathway is in energy communication with an energy emitter which directs energy into the energy pathway and with an energy receiver which measures energy transmission through the energy pathway; and wherein changes in the transmission of energy through energy pathways are analyzed to measure the configuration and motion of body joints.

In an example, electrical and/or electromagnetic energy can be emitted by an energy emitter, transmitted through an energy pathway, and received by an energy receiver. In an example, light energy can be emitted by an energy emitter, transmitted through an energy pathway, and received by an energy receiver. In an example, sonic energy can be emitted by an energy emitter, transmitted through an energy pathway, and received by an energy receiver. In an example, the results of analysis of energy transmission through a first energy pathway can trigger energy transmission through a second energy pathway. In an example, energy transmission through the second energy pathway can require more energy than energy transmission through the first energy pathway.

In an example, an energy pathway can comprise a loop which spans a body joint, wherein ends of the loop are both proximal relative to the joint or are both distal relative to the joint. In an example, an energy pathway can comprise a loop which spans a body joint, wherein an energy emitter in communication with the pathway and an energy receiver in communication with the pathway are both proximal relative to the joint or are both distal relative to the joint. In an example, a first subset of the energy pathways can be substantively parallel to a longitudinal axis of a body joint and a second subset of the energy pathways can be partially-helical around a body member containing the body joint. In an example, a garment or article of clothing can further comprise inertial motion sensors located proximally and distally relative to the body joint, wherein data from the energy pathways and data from the inertial motion sensors are jointly analyzed to measure and/or recognize joint configuration and/or motion.

I claim:

1. An upper-body garment or article of clothing for measuring body configuration and motion comprising:
an upper-body garment or article of clothing worn by a person, wherein the garment or article of clothing further comprises;
a set of energy pathways which span the person's right elbow;
a set of proximally-diverging energy pathways which span the person's right shoulder;
a set of energy pathways which span the person's left elbow;
a set of proximally-diverging energy pathways which span the person's left shoulder;
a set of energy pathways which span a portion of the person's torso or back;
a plurality of energy emitters; and
a plurality of energy receivers;
wherein each energy pathway is in energy communication with an energy emitter which directs energy into the energy pathway and with an energy receiver which measures energy transmission through the energy pathway;
and wherein changes in the transmission of energy through energy pathways are analyzed to measure the configuration and motion of body joints.

2. The garment or article of clothing in claim 1 wherein electrical and/or electromagnetic energy is emitted by an energy emitter, transmitted through an energy pathway, and received by an energy receiver.

3. The garment or article of clothing in claim 1 wherein light energy is emitted by an energy emitter, transmitted through an energy pathway, and received by an energy receiver.

4. The garment or article of clothing in claim 1 wherein sonic energy is emitted by an energy emitter, transmitted through an energy pathway, and received by an energy receiver.

5. The garment or article of clothing in claim 1 wherein the results of analysis of energy transmission through a first energy pathway trigger energy transmission through a second energy pathway.

6. The garment or article of clothing in claim 5 wherein energy transmission through the second energy pathway requires more energy than energy transmission through the first energy pathway.

7. The garment or article of clothing in claim 1 wherein an energy pathway comprises a loop which spans a body joint, and wherein ends of the loop are both proximal relative to the joint or are both distal relative to the joint.

8. The garment or article of clothing in claim 1 wherein an energy pathway comprises a loop which spans a body joint, and wherein an energy emitter in communication with the pathway and an energy receiver in communication with the pathway are both proximal relative to the joint or are both distal relative to the joint.

9. The garment or article of clothing in claim 1 wherein a first subset of the energy pathways are substantively parallel to a longitudinal axis of a body joint and a second subset of the energy pathways are partially-helical around a body member containing the body joint.

10. The garment or article of clothing in claim 1 wherein the garment or article of clothing further comprises inertial motion sensors located proximally and distally relative to the body joint, and wherein data from the energy pathways and data from the inertial motion sensors are jointly analyzed to measure and/or recognize joint configuration and/or motion.

11. A lower-body garment or article of clothing for measuring body configuration and motion comprising:
a lower-body garment or article of clothing worn by a person, wherein the garment or article of clothing further comprises;
a set of energy pathways which span the person's right knee;
a set of proximally-diverging energy pathways which span the person's right hip;
a set of energy pathways which span the person's left knee;
a set of proximally-diverging energy pathways which span the person's left hip;
a plurality of energy emitters; and
a plurality of energy receivers;
wherein each energy pathway is in energy communication with an energy emitter which directs energy into the energy pathway and with an energy receiver which measures energy transmission through the energy pathway;
and wherein changes in the transmission of energy through energy pathways are analyzed to measure the configuration and motion of body joints.

12. The garment or article of clothing in claim 11 wherein electrical and/or electromagnetic energy is emitted by an energy emitter, transmitted through an energy pathway, and received by an energy receiver.

13. The garment or article of clothing in claim 11 wherein light energy is emitted by an energy emitter, transmitted through an energy pathway, and received by an energy receiver.

14. The garment or article of clothing in claim 11 wherein sonic energy is emitted by an energy emitter, transmitted through an energy pathway, and received by an energy receiver.

15. The garment or article of clothing in claim 11 wherein the results of analysis of energy transmission through a first energy pathway trigger energy transmission through a second energy pathway.

16. The garment or article of clothing in claim 15 wherein energy transmission through the second energy pathway requires more energy than energy transmission through the first energy pathway.

17. The garment or article of clothing in claim 11 wherein an energy pathway comprises a loop which spans a body joint, and wherein ends of the loop are both proximal relative to the joint or are both distal relative to the joint.

18. The garment or article of clothing in claim 11 wherein an energy pathway comprises a loop which spans a body joint, and wherein an energy emitter in communication with the pathway and an energy receiver in communication with the pathway are both proximal relative to the joint or are both distal relative to the joint.

19. The garment or article of clothing in claim 11 wherein a first subset of the energy pathways are substantively parallel to a longitudinal axis of a body joint and a second subset of the energy pathways are partially-helical around a body member containing the body joint.

20. The garment or article of clothing in claim 11 wherein the garment or article of clothing further comprises inertial motion sensors located proximally and distally relative to the body joint, and wherein data from the energy pathways and data from the inertial motion sensors are jointly analyzed to measure and/or recognize joint configuration and/or motion.
